(19) United States
(12) Patent Application Publication
(10) Pub. No.: US 2023/0111568 A1
(43) Pub. Date: Apr. 13, 2023

(54) SOLID DISPERSIONS OF RIFAXIMIN FOR THE TREATMENT OF OVERT HEPATIC ENCEPHALOPATHY

(71) Applicant: Bausch Health Ireland Limited, Dublin (IE)

(72) Inventors: Zeev Heimanson, Calabasas, CA (US); Robert J. Israel, Suffern, NY (US); Thejasvi Ramakrishna, Flemington, NJ (US); Jason Lamar Vittitow, Flanders, NJ (US)

(21) Appl. No.: 17/913,844

(22) PCT Filed: Mar. 24, 2021
(86) PCT No.: PCT/EP2021/057647

§ 371 Date: Sep. 23, 2022

Related U.S. Application Data
(60) Provisional application No. 63/158,784, filed on Mar. 9, 2021.
(60) Provisional application No. 62/994,013, filed on Mar. 24, 2020.

(51) Int. Cl. A61K 31/437; A61P 1/16; A61P 25/28; A61K 31/7016; A61K 9/20; A61K 9/28

(52) U.S. Cl. CPC A61K 31/437; A61P 1/16; A61P 25/28; A61K 31/7016; A61K 9/2054; A61K 9/2031; A61K 9/2009; A61K 9/282; A61K 9/2013

(57) ABSTRACT

Provided herein are solid dispersions comprising rifaximin and methods of using the same for the treatment of Overt Hepatic Encephalopathy and complications associated with cirrhosis.

### RELATED APPLICATIONS

This application claims priority to U.S. Provisional Application No. 62/994,013, filed Mar. 24, 2020 and U.S. Provisional Application No. 63/158,784, filed Mar. 9, 2021, the entire contents of each of which are incorporated herein by reference.

### BACKGROUND

The incidence of liver disease is on the rise and will continue to be a major health burden. Alcohol is the major cause of liver disease, but metabolic syndrome as a cause of nonalcoholic fatty acid liver disease may soon surpass alcohol as the major cause of CLD. Cirrhosis is a major cause of much of the chronic liver disease (CLD) in at least the U.S., and is the number one cause of CLD related deaths and cirrhosis and CLD combined is the 11th leading cause of death in the U.S. Mortality rates from complications of cirrhosis using data from the Nationwide Inpatient Sample (NIS) database from 1998 to 2006 were estimated to be approximately 8%, 18%, 10%, and 45% for ascites, hepatic encephalopathy (HE), variceal bleeding, and hepatorenal syndrome (HRS), respectively. See e.g., Mol Pharm. 2011; 8:1573-1581. Similarly, other studies have demonstrated that the in-hospital mortality of patients with spontaneous bacterial peritonitis (SBP) ranges from 10-50%. See e.g., Hepatology, 1993 February; 17(2):251-7; J Hepatol, 2004 May; 40(5):823-30.

The management of cirrhosis and its complications is based on disease severity and whether or not complications have developed (i.e., decompensated disease). The development of esophageal variceal bleeding (EVB), ascites, spontaneous bacterial peritonitis (SBP), HE or hepatorenal syndrome (HRS) has a profound impact on prognosis. Despite current medical therapies for EVB, ascites, SBP, and HE, patients with compensated disease who develop one of these complications have a five-year survival rate of 20% to 50% (Gastroenterology 1987; 93: 234-241; Gastroenterology. 1997; 112:463-472). The survival rate of patients who develop SBP or HRS is particularly poor. For SBP, less than half will survive 1-year; the median survival rate for patients with type I HRS is less than 2 weeks (Gastroenterology. 1993; 104:1133-8; Gastroenterology 1993; 105: 229-236). The 30-day and 3-month mortality rates in the SBP group were 26.1 and 50.7% respectively with the 30-day mortality rate for HRS being 46.72%

Given the continued escalation of liver disease and related conditions, a need exists in the field for new therapies and treatment methodologies.

### SUMMARY

As described herein, it has been surprisingly discovered that an immediate release (IR) formulation of rifaximin as a soluble solid dispersion (SSD), having a 40 mg dose of rifaximin, in combination with lactulose was superior to lactulose in treating patients having Overt Hepatic Encephalopathy (OHE). In view of these findings, the invention described herein provides, for example, therapies for OHE, including preventing or delaying the onset of OHE and preventing or delaying complications of liver cirrhosis in subjects, such as those with early decompensation.

As a further example, the invention disclosed herein includes one or more of reducing the risk of OHE, reducing the severity of OHE, reducing the duration of OHE, and reducing HE related hospitalization comprising the administration of at least one rifaximin SSD composition described herein.

For example, the superiority of rifaximin SSD IR (40 mg) was demonstrated by (1) a reduction in time to OHE resolution (as compared to lactulose alone), which was measured by Hepatic Encephalopathy Grading Instrument (HEGI) score; and (2) a reduction in time to improvement in HEGI score (as compared to lactulose alone).

In an embodiment, the invention described herein includes a method of treating Overt Hepatic Encephalopathy (OHE) in a patient in need thereof comprising administering at least one 40 mg rifaximin SSD IR tablet to the patient until OHE symptoms resolve.

In an embodiment, the invention described herein includes a method of treating Overt Hepatic Encephalopathy (OHE) in a patient in need thereof comprising administering at least one 40 mg rifaximin SSD IR tablet to the patient for at least one day, or at least two days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days.

In an embodiment, the invention described herein includes a method of treating Overt Hepatic Encephalopathy (OHE) in a patient in need thereof comprising administering a 40 mg rifaximin SSD IR tablet QD to the patient for at least one day, or at least two days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days.

In an embodiment, the invention described herein includes a method of treating Overt Hepatic Encephalopathy (OHE) in a patient in need thereof comprising administering a 40 mg rifaximin SSD IR tablet BID to the patient for at least one day, or at least two days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days.

In an embodiment, the invention described herein includes a method of treating Overt Hepatic Encephalopathy (OHE) in a patient in need thereof comprising administering a 40 mg rifaximin SSD IR tablet TID to the patient for at least one day, or at least two days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days.

In an embodiment, the invention described herein includes a method of treating Overt Hepatic Encephalopathy (OHE) in a patient in need thereof comprising administering lactulose and at least one 40 mg rifaximin SSD IR tablet to the patient until OHE symptoms resolve.

In an embodiment, the invention described herein includes a method of treating Overt Hepatic Encephalopathy (OHE) in a patient in need thereof comprising administering lactulose and at least one 40 mg rifaximin SSD IR tablet to the patient for at least one day, or at least two days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days.

In an embodiment, the invention described herein includes a method of treating Overt Hepatic Encephalopathy (OHE) in a patient in need thereof comprising administering lactulose and a 40 mg rifaximin SSD IR tablet QD to the patient for at least one day, or at least two days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days.

In an embodiment, the invention described herein includes a method of treating Overt Hepatic Encephalopathy (OHE) in a patient in need thereof comprising administering lactulose and a 40 mg rifaximin SSD IR tablet BID to the patient for at least one day, or at least two days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days.

In an embodiment, the invention described herein includes a method of treating Overt Hepatic Encephalopathy (OHE) in a patient in need thereof comprising administering lactulose and a 40 mg rifaximin SSD IR tablet TID to the patient for at least one day, or at least two days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days.

In an embodiment, the invention described herein includes a method of treating Overt Hepatic Encephalopathy (OHE) in a patient in need thereof comprising administering at least one 40 mg rifaximin SSD IR tablet to the patient until the patient's Hepatic Encephalopathy Grading Instrument (HEGI) score is reduced by at least one grade, or at least two grades, or at least three grades.

In an embodiment, the invention described herein includes a method of treating Overt Hepatic Encephalopathy (OHE) in a patient in need thereof comprising administering at least one 40 mg rifaximin SSD IR tablet to the patient until the patient's Hepatic Encephalopathy Grading Instrument (HEGI) score is reduced from grade 4 to grade 3 or from grade 3 to grade 2.

In an embodiment, the invention described herein includes a method of treating Overt Hepatic Encephalopathy (OHE) in a patient in need thereof comprising administering a 40 mg rifaximin SSD IR tablet QD to the patient until the patient's Hepatic Encephalopathy Grading Instrument (HEGI) score is reduced by at least one grade, or at least two grades, or at least three grades.

In an embodiment, the invention described herein includes a method of treating Overt Hepatic Encephalopathy (OHE) in a patient in need thereof comprising administering a 40 mg rifaximin SSD IR tablet QD to the patient until the patient's Hepatic Encephalopathy Grading Instrument (HEGI) score is reduced from grade 4 to grade 3 or from grade 3 to grade 2.

In an embodiment, the invention described herein includes a method of treating Overt Hepatic Encephalopathy (OHE) in a patient in need thereof comprising administering a 40 mg rifaximin SSD IR tablet BID to the patient until the patient's Hepatic Encephalopathy Grading Instrument (REGI) score is reduced by at least one grade, or at least two grades, or at least three grades.

In an embodiment, the invention described herein includes a method of treating Overt Hepatic Encephalopathy (OHE) in a patient in need thereof comprising administering a 40 mg rifaximin SSD IR tablet BID to the patient until the patient's Hepatic Encephalopathy Grading Instrument (REGI) score is reduced from grade 4 to grade 3 or from grade 3 to grade 2.

In an embodiment, the invention described herein includes a method of treating Overt Hepatic Encephalopathy (OHE) in a patient in need thereof comprising administering a 40 mg rifaximin SSD IR tablet TID to the patient until the patient's Hepatic Encephalopathy Grading Instrument (REGI) score is reduced by at least one grade, or at least two grades, or at least three grades.

In an embodiment, the invention described herein includes a method of treating Overt Hepatic Encephalopathy (OHE) in a patient in need thereof comprising administering a 40 mg rifaximin SSD IR tablet TID to the patient until the patient's Hepatic Encephalopathy Grading Instrument (REGI) score is reduced from grade 4 to grade 3 or from grade 3 to grade 2.

In an embodiment, the invention described herein includes a method of treating Overt Hepatic Encephalopathy (OHE) in a patient in need thereof comprising concomitantly administering lactulose and at least one 40 mg rifaximin SSD IR tablet to the patient until the patient's Hepatic Encephalopathy Grading Instrument (REGI) score is reduced by at least one grade, or at least two grades, or at least three grades.

In an embodiment, the invention described herein includes a method of treating Overt Hepatic Encephalopathy (OHE) in a patient in need thereof comprising administering lactulose and at least one 40 mg rifaximin SSD IR tablet to the patient until the patient's Hepatic Encephalopathy Grading Instrument (REGI) score is reduced from grade 4 to grade 3 or from grade 3 to grade 2.

In an embodiment, the invention described herein includes a method of treating Overt Hepatic Encephalopathy (OHE) in a patient in need thereof comprising administering lactulose and a 40 mg rifaximin SSD IR tablet QD to the patient until the patient's Hepatic Encephalopathy Grading Instrument (REGI) score is reduced by at least one grade, or at least two grades, or at least three grades.

In an embodiment, the invention described herein includes a method of treating Overt Hepatic Encephalopathy (OHE) in a patient in need thereof comprising administering lactulose and a 40 mg rifaximin SSD IR tablet QD to the patient until the patient's Hepatic Encephalopathy Grading Instrument (REGI) score is reduced from grade 4 to grade 3 or from grade 3 to grade 2.

In an embodiment, the invention described herein includes a method of treating Overt Hepatic Encephalopathy (OHE) in a patient in need thereof comprising administering lactulose and a 40 mg rifaximin SSD IR tablet BID to the patient until the patient's Hepatic Encephalopathy Grading Instrument (REGI) score is reduced by at least one grade, or at least two grades, or at least three grades.

In an embodiment, the invention described herein includes a method of treating Overt Hepatic Encephalopathy (OHE) in a patient in need thereof comprising administering lactulose and a 40 mg rifaximin SSD IR tablet BID to the patient until the patient's Hepatic Encephalopathy Grading Instrument (REGI) score is reduced from grade 4 to grade 3 or from grade 3 to grade 2.

In an embodiment, the invention described herein includes a method of treating Overt Hepatic Encephalopathy (OHE) in a patient in need thereof comprising administering lactulose and a 40 mg rifaximin SSD IR tablet TID to the patient until the patient's Hepatic Encephalopathy Grading Instrument (REGI) score is reduced by at least one grade, or at least two grades, or at least three grades.

In an embodiment, the invention described herein includes a method of treating Overt Hepatic Encephalopathy (OHE) in a patient in need thereof comprising administering lactulose and a 40 mg rifaximin SSD IR tablet TID to the patient until the patient's Hepatic Encephalopathy Grading Instrument (REGI) score is reduced from grade 4 to grade 3 or from grade 3 to grade 2.

In an embodiment, the invention described herein includes a method of treating Overt Hepatic Encephalopathy (OHE) in a patient in need thereof comprising administering at least one 40 mg rifaximin SSD IR tablet to the patient until the patient's West Haven Criteria (WHC) score is reduced by at least one grade, or at least two grades, or at least three grades.

In an embodiment, the invention described herein includes a method of treating Overt Hepatic Encephalopathy (OHE) in a patient in need thereof comprising administering at least one 40 mg rifaximin SSD IR tablet to the patient until the patient's West Haven Criteria (WHC) score is reduced from grade 4 to grade 3 or from grade 3 to grade 2 or from grade 2 to grade 1 or from grade 1 to grade 0.

In an embodiment, the invention described herein includes a method of treating Overt Hepatic Encephalopathy (OHE) in a patient in need thereof comprising administering a 40 mg rifaximin SSD IR tablet QD to the patient until the patient's West Haven Criteria (WHC) score is reduced by at least one grade, or at least two grades, or at least three grades.

In an embodiment, the invention described herein includes a method of treating Overt Hepatic Encephalopathy (OHE) in a patient in need thereof comprising administering a 40 mg rifaximin SSD IR tablet QD to the patient until the patient's West Haven Criteria (WHC) score is reduced from grade 4 to grade 3 or from grade 3 to grade 2 or from grade 2 to grade 1 or from grade 1 to grade 0.

In an embodiment, the invention described herein includes a method of treating Overt Hepatic Encephalopathy (OHE) in a patient in need thereof comprising administering a 40 mg rifaximin SSD IR tablet BID to the patient until the patient's West Haven Criteria (WHC) score is reduced by at least one grade, or at least two grades, or at least three grades.

In an embodiment, the invention described herein includes a method of treating Overt Hepatic Encephalopathy (OHE) in a patient in need thereof comprising administering a 40 mg rifaximin SSD IR tablet BID to the patient until the patient's West Haven Criteria (WHC) score is reduced from grade 4 to grade 3 or from grade 3 to grade 2 or from grade 2 to grade 1 or from grade 1 to grade 0.

In an embodiment, the invention described herein includes a method of treating Overt Hepatic Encephalopathy (OHE) in a patient in need thereof comprising administering a 40 mg rifaximin SSD IR tablet TID to the patient until the patient's West Haven Criteria (WHC) score is reduced by at least one grade, or at least two grades, or at least three grades.

In an embodiment, the invention described herein includes a method of treating Overt Hepatic Encephalopathy (OHE) in a patient in need thereof comprising administering a 40 mg rifaximin SSD IR tablet TID to the patient until the patient's West Haven Criteria (WHC) score is reduced from grade 4 to grade 3 or from grade 3 to grade 2 or from grade 2 to grade 1 or from grade 1 to grade 0.

In an embodiment, the invention described herein includes a method of treating Overt Hepatic Encephalopathy (OHE) in a patient in need thereof comprising administering lactulose and at least one 40 mg rifaximin SSD IR tablet to the patient until the patient's West Haven Criteria (WHC) score is reduced by at least one grade, or at least two grades, or at least three grades.

In an embodiment, the invention described herein includes a method of treating Overt Hepatic Encephalopathy (OHE) in a patient in need thereof comprising administering lactulose and at least one 40 mg rifaximin SSD IR tablet to the patient until the patient's West Haven Criteria (WHC) score is reduced from grade 4 to grade 3 or from grade 3 to grade 2 or from grade 2 to grade 1 or from grade 1 to grade 0.

In an embodiment, the invention described herein includes a method of treating Overt Hepatic Encephalopathy (OHE) in a patient in need thereof comprising administering lactulose and a 40 mg rifaximin SSD IR tablet QD to the patient until the patient's West Haven Criteria (WHC) score is reduced by at least one grade, or at least two grades, or at least three grades.

In an embodiment, the invention described herein includes a method of treating Overt Hepatic Encephalopathy (OHE) in a patient in need thereof comprising administering lactulose and a 40 mg rifaximin SSD IR tablet QD to the patient until the patient's West Haven Criteria (WHC) score is reduced from grade 4 to grade 3 or from grade 3 to grade 2 or from grade 2 to grade 1 or from grade 1 to grade 0.

In an embodiment, the invention described herein includes a method of treating Overt Hepatic Encephalopathy (OHE) in a patient in need thereof comprising administering lactulose and a 40 mg rifaximin SSD IR tablet BID to the patient until the patient's West Haven Criteria (WHC) score is reduced by at least one grade, or at least two grades, or at least three grades.

In an embodiment, the invention described herein includes a method of treating Overt Hepatic Encephalopathy (OHE) in a patient in need thereof comprising administering lactulose and a 40 mg rifaximin SSD IR tablet BID to the patient until the patient's West Haven Criteria (WHC) score is reduced from grade 4 to grade 3 or from grade 3 to grade 2 or from grade 2 to grade 1 or from grade 1 to grade 0.

In an embodiment, the invention described herein includes a method of treating Overt Hepatic Encephalopathy (OHE) in a patient in need thereof comprising administering lactulose and a 40 mg rifaximin SSD IR tablet TID to the patient until the patient's West Haven Criteria (WHC) score is reduced by at least one grade, or at least two grades, or at least three grades.

In an embodiment, the invention described herein includes a method of treating Overt Hepatic Encephalopathy (OHE) in a patient in need thereof comprising administering lactulose and a 40 mg rifaximin SSD IR tablet TID to the patient until the patient's West Haven Criteria (WHC) score is reduced from grade 4 to grade 3 or from grade 3 to grade 2 or from grade 2 to grade 1 or from grade 1 to grade 0.

In an embodiment, the invention described herein includes a method of treating Overt Hepatic Encephalopathy (OHE) in a patient in need thereof comprising administering a daily dose of 40 mg rifaximin in a rifaximin SSD IR tablet dosage form to the patient for at least one day, or at least two days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days.

In an embodiment, the invention described herein includes a method of treating Overt Hepatic Encephalopathy (OHE) in a patient in need thereof comprising administering a daily dose of 80 mg rifaximin in a rifaximin SSD IR tablet dosage form to the patient for at least one day, or at least two days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days.

In an embodiment, the invention described herein includes a method of treating Overt Hepatic Encephalopathy (OHE) in a patient in need thereof comprising administering a daily dose of 120 mg rifaximin in a rifaximin SSD IR tablet dosage form to the patient for at least one day, or at least two days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days.

In an embodiment, the invention described herein includes a method of treating Overt Hepatic Encephalopathy (OHE) in a patient in need thereof comprising administering lactulose and a daily dose of 40 mg rifaximin in a rifaximin SSD IR tablet dosage form to the patient until OHE symptoms resolve.

In an embodiment, the invention described herein includes a method of treating Overt Hepatic Encephalopathy (OHE) in a patient in need thereof comprising administering lactulose and a daily dose of 40 mg rifaximin in a rifaximin SSD IR tablet dosage form to the patient for at least one day, or at least two days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days.

In an embodiment, the invention described herein includes a method of treating Overt Hepatic Encephalopathy (OHE) in a patient in need thereof comprising administering lactulose and a daily dose of 80 mg rifaximin in a rifaximin SSD IR tablet dosage form to the patient for at least one day, or at least two days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days.

In an embodiment, the invention described herein includes a method of treating Overt Hepatic Encephalopathy (OHE) in a patient in need thereof comprising administering lactulose and a daily dose of 120 mg rifaximin in a rifaximin SSD IR tablet dosage form to the patient for at least one day, or at least two days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days.

In an embodiment, the invention described herein includes a method of treating Overt Hepatic Encephalopathy (OHE) in a patient in need thereof comprising administering a daily dose of 40 mg rifaximin in a rifaximin SSD IR tablet dosage form to the patient until the patient's Hepatic Encephalopathy Grading Instrument (HEGI) score is reduced by at least one grade, or at least two grades, or at least three grades.

In an embodiment, the invention described herein includes a method of treating Overt Hepatic Encephalopathy (OHE) in a patient in need thereof comprising administering a daily dose of 40 mg rifaximin in a rifaximin SSD IR tablet dosage form to the patient until the patient's Hepatic Encephalopathy Grading Instrument (HEGI) score is reduced from grade 4 to grade 3 or from grade 3 to grade 2.

In an embodiment, the invention described herein includes a method of treating Overt Hepatic Encephalopathy (OHE) in a patient in need thereof comprising administering a daily dose of 80 mg rifaximin in a rifaximin SSD IR tablet dosage form to the patient until the patient's Hepatic Encephalopathy Grading Instrument (HEGI) score is reduced by at least one grade, or at least two grades, or at least three grades.

In an embodiment, the invention described herein includes a method of treating Overt Hepatic Encephalopathy (OHE) in a patient in need thereof comprising administering a daily dose of 80 mg rifaximin in a rifaximin SSD IR tablet dosage form to the patient until the patient's Hepatic Encephalopathy Grading Instrument (HEGI) score is reduced from grade 4 to grade 3 or from grade 3 to grade 2.

In an embodiment, the invention described herein includes a method of treating Overt Hepatic Encephalopathy (OHE) in a patient in need thereof comprising administering a daily dose of 120 mg rifaximin in a rifaximin SSD IR tablet dosage form to the patient until the patient's Hepatic Encephalopathy Grading Instrument (HEGI) score is reduced by at least one grade, or at least two grades, or at least three grades.

In an embodiment, the invention described herein includes a method of treating Overt Hepatic Encephalopathy (OHE) in a patient in need thereof comprising administering a daily dose of 120 mg rifaximin in a rifaximin SSD IR tablet dosage form to the patient until the patient's Hepatic Encephalopathy Grading Instrument (HEGI) score is reduced from grade 4 to grade 3 or from grade 3 to grade 2.

In an embodiment, the invention described herein includes a method of treating Overt Hepatic Encephalopathy (OHE) in a patient in need thereof comprising administering lactulose and a daily dose of 40 mg rifaximin in a rifaximin SSD IR tablet dosage form to the patient until the patient's Hepatic Encephalopathy Grading Instrument (HEGI) score is reduced by at least one grade, or at least two grades, or at least three grades.

In an embodiment, the invention described herein includes a method of treating Overt Hepatic Encephalopathy (OHE) in a patient in need thereof comprising administering lactulose and a daily dose of 40 mg rifaximin in a rifaximin SSD IR tablet dosage form to the patient until the patient's Hepatic Encephalopathy Grading Instrument (HEGI) score is reduced from grade 4 to grade 3 or from grade 3 to grade 2.

In an embodiment, the invention described herein includes a method of treating Overt Hepatic Encephalopathy (OHE) in a patient in need thereof comprising administering lactulose and a daily dose of 80 mg rifaximin in a rifaximin SSD IR tablet dosage form to the patient until the patient's Hepatic Encephalopathy Grading Instrument (HEGI) score is reduced by at least one grade, or at least two grades, or at least three grades.

In an embodiment, the invention described herein includes a method of treating Overt Hepatic Encephalopathy (OHE) in a patient in need thereof comprising administering lactulose and a daily dose of 80 mg rifaximin in a rifaximin SSD IR tablet dosage form to the patient until the patient's Hepatic Encephalopathy Grading Instrument (HEGI) score is reduced from grade 4 to grade 3 or from grade 3 to grade 2.

In an embodiment, the invention described herein includes a method of treating Overt Hepatic Encephalopathy (OHE) in a patient in need thereof comprising administering lactulose and a daily dose of 120 mg rifaximin in a rifaximin SSD IR tablet dosage form to the patient until the patient's Hepatic Encephalopathy Grading Instrument (HEGI) score is reduced by at least one grade, or at least two grades, or at least three grades.

In an embodiment, the invention described herein includes a method of treating Overt Hepatic Encephalopathy (OHE) in a patient in need thereof comprising administering lactulose and a daily dose of 120 mg rifaximin in a rifaximin SSD IR tablet dosage form to the patient until the patient's Hepatic Encephalopathy Grading Instrument (HEGI) score is reduced from grade 4 to grade 3 or from grade 3 to grade 2.

In an embodiment, the invention described herein includes a method of treating Overt Hepatic Encephalopathy (OHE) in a patient in need thereof comprising administering a daily dose of 40 mg rifaximin in a rifaximin SSD IR tablet dosage form to the patient until the patient's West Haven Criteria (WHC) score is reduced by at least one grade, or at least two grades, or at least three grades.

In an embodiment, the invention described herein includes a method of treating Overt Hepatic Encephalopathy (OHE) in a patient in need thereof comprising administering a daily dose of 40 mg rifaximin in a rifaximin SSD IR tablet dosage form to the patient until the patient's West Haven Criteria (WHC) score is reduced from grade 4 to grade 3 or from grade 3 to grade 2 or from grade 2 to grade 1 or from grade 1 to grade 0.

In an embodiment, the invention described herein includes a method of treating Overt Hepatic Encephalopathy (OHE) in a patient in need thereof comprising administering a daily dose of 80 mg rifaximin in a rifaximin SSD IR tablet dosage form to the patient until the patient's West Haven Criteria (WHC) score is reduced by at least one grade, or at least two grades, or at least three grades.

In an embodiment, the invention described herein includes a method of treating Overt Hepatic Encephalopathy (OHE) in a patient in need thereof comprising administering a daily dose of 80 mg rifaximin in a rifaximin SSD IR tablet dosage form to the patient until the patient's West Haven Criteria (WHC) score is reduced from grade 4 to grade 3 or from grade 3 to grade 2 or from grade 2 to grade 1 or from grade 1 to grade 0.

In an embodiment, the invention described herein includes a method of treating Overt Hepatic Encephalopathy (OHE) in a patient in need thereof comprising administering a daily dose of 120 mg rifaximin in a rifaximin SSD IR tablet dosage form to the patient until the patient's West Haven Criteria (WHC) score is reduced by at least one grade, or at least two grades, or at least three grades.

In an embodiment, the invention described herein includes a method of treating Overt Hepatic Encephalopathy (OHE) in a patient in need thereof comprising administering a daily dose of 120 mg rifaximin in a rifaximin SSD IR tablet dosage form to the patient until the patient's West Haven Criteria (WHC) score is reduced from grade 4 to grade 3 or from grade 3 to grade 2 or from grade 2 to grade 1 or from grade 1 to grade 0.

In an embodiment, the invention described herein includes a method of treating Overt Hepatic Encephalopathy (OHE) in a patient in need thereof comprising administering lactulose and a daily dose of 40 mg rifaximin in a rifaximin SSD IR tablet dosage form to the patient until the patient's West Haven Criteria (WHC) score is reduced by at least one grade, or at least two grades, or at least three grades.

In an embodiment, the invention described herein includes a method of treating Overt Hepatic Encephalopathy (OHE) in a patient in need thereof comprising administering lactulose and a daily dose of 40 mg rifaximin in a rifaximin SSD IR tablet dosage form to the patient until the patient's West Haven Criteria (WHC) score is reduced from grade 4 to grade 3 or from grade 3 to grade 2 or from grade 2 to grade 1 or from grade 1 to grade 0.

In an embodiment, the invention described herein includes a method of treating Overt Hepatic Encephalopathy (OHE) in a patient in need thereof comprising administering lactulose and a daily dose of 80 mg rifaximin in a rifaximin SSD IR tablet dosage form to the patient until the patient's West Haven Criteria (WHC) score is reduced by at least one grade, or at least two grades, or at least three grades.

In an embodiment, the invention described herein includes a method of treating Overt Hepatic Encephalopathy (OHE) in a patient in need thereof comprising administering lactulose and a daily dose of 80 mg rifaximin in a rifaximin SSD IR tablet dosage form to the patient until the patient's West Haven Criteria (WHC) score is reduced from grade 4 to grade 3 or from grade 3 to grade 2 or from grade 2 to grade 1 or from grade 1 to grade 0.

In an embodiment, the invention described herein includes a method of treating Overt Hepatic Encephalopathy (OHE) in a patient in need thereof comprising administering lactulose and a daily dose of 120 mg rifaximin in a rifaximin SSD IR tablet dosage form to the patient until the patient's West Haven Criteria (WHC) score is reduced by at least one grade, or at least two grades, or at least three grades.

In an embodiment, the invention described herein includes a method of treating Overt Hepatic Encephalopathy (OHE) in a patient in need thereof comprising administering lactulose and a daily dose of 120 mg rifaximin in a rifaximin SSD IR tablet dosage form to the patient until the patient's West Haven Criteria (WHC) score is reduced from grade 4 to grade 3 or from grade 3 to grade 2 or from grade 2 to grade 1 or from grade 1 to grade 0.

Also disclosed are methods of treating Overt Hepatic Encephalopathy (OHE) in a patient in need thereof comprising administering to the patient at least one rifaximin SSD IR tablet.

In an embodiment, the invention described herein includes a method of treating Overt Hepatic Encephalopathy (OHE) in a patient in need thereof comprising administering at least one rifaximin SSD IR composition (e.g., tablet) to the patient until OHE symptoms resolve.

In an embodiment, the invention described herein includes a method of treating Overt Hepatic Encephalopathy (OHE) in a patient in need thereof comprising administering at least one rifaximin SSD IR composition (e.g., tablet) to the patient for at least one day, or at least two days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days.

In an embodiment, the invention described herein includes a method of treating Overt Hepatic Encephalopathy (OHE) in a patient in need thereof comprising administering a rifaximin SSD IR composition (e.g., tablet) QD to the patient for at least one day, or at least two days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days.

In an embodiment, the invention described herein includes a method of treating Overt Hepatic Encephalopathy (OHE) in a patient in need thereof comprising administering a rifaximin SSD IR composition (e.g., tablet) BID to the patient for at least one day, or at least two days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days.

In an embodiment, the invention described herein includes a method of treating Overt Hepatic Encephalopathy (OHE) in a patient in need thereof comprising administering a rifaximin SSD IR composition (e.g., tablet) TID to the patient for at least one day, or at least two days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days.

In an embodiment, the invention described herein includes a method of treating Overt Hepatic Encephalopathy (OHE) in a patient in need thereof comprising administering lactulose and at least one rifaximin SSD IR composition (e.g., tablet) to the patient until OHE symptoms resolve.

In an embodiment, the invention described herein includes a method of treating Overt Hepatic Encephalopathy (OHE) in a patient in need thereof comprising administering lactulose and at least one rifaximin SSD IR composition (e.g., tablet) to the patient for at least one day, or at least two days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days.

In an embodiment, the invention described herein includes a method of treating Overt Hepatic Encephalopathy (OHE) in a patient in need thereof comprising administering lactulose and a rifaximin SSD IR composition (e.g., tablet) QD to the patient for at least one day, or at least two days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days.

In an embodiment, the invention described herein includes a method of treating Overt Hepatic Encephalopathy (OHE) in a patient in need thereof comprising administering lactulose and a rifaximin SSD IR composition (e.g., tablet) BID to the patient for at least one day, or at least two days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days.

In an embodiment, the invention described herein includes a method of treating Overt Hepatic Encephalopathy (OHE) in a patient in need thereof comprising administering lactulose and a rifaximin SSD IR composition (e.g., tablet) TID to the patient for at least one day, or at least two days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days.

In an embodiment, the invention described herein includes a method of treating Overt Hepatic Encephalopathy (OHE) in a patient in need thereof comprising administering at least one rifaximin SSD IR composition (e.g., tablet) to the patient until the patient's Hepatic Encephalopathy Grading Instrument (REGI) score is reduced by at least one grade, or at least two grades, or at least three grades.

In an embodiment, the invention described herein includes a method of treating Overt Hepatic Encephalopathy (OHE) in a patient in need thereof comprising administering at least one rifaximin SSD IR composition (e.g., tablet) to the patient until the patient's Hepatic Encephalopathy Grading Instrument (REGI) score is reduced from grade 4 to grade 3 or from grade 3 to grade 2.

In an embodiment, the invention described herein includes a method of treating Overt Hepatic Encephalopathy (OHE) in a patient in need thereof comprising administering a rifaximin SSD IR composition (e.g., tablet) QD to the patient until the patient's Hepatic Encephalopathy Grading Instrument (REGI) score is reduced by at least one grade, or at least two grades, or at least three grades.

In an embodiment, the invention described herein includes a method of treating Overt Hepatic Encephalopathy (OHE) in a patient in need thereof comprising administering a rifaximin SSD IR composition (e.g., tablet) QD to the patient until the patient's Hepatic Encephalopathy Grading Instrument (REGI) score is reduced from grade 4 to grade 3 or from grade 3 to grade 2.

In an embodiment, the invention described herein includes a method of treating Overt Hepatic Encephalopathy (OHE) in a patient in need thereof comprising administering a rifaximin SSD IR composition (e.g., tablet) BID to the patient until the patient's Hepatic Encephalopathy Grading Instrument (HEGI) score is reduced by at least one grade, or at least two grades, or at least three grades.

In an embodiment, the invention described herein includes a method of treating Overt Hepatic Encephalopathy (OHE) in a patient in need thereof comprising administering a rifaximin SSD IR composition (e.g., tablet) BID to the patient until the patient's Hepatic Encephalopathy Grading Instrument (HEGI) score is reduced from grade 4 to grade 3 or from grade 3 to grade 2.

In an embodiment, the invention described herein includes a method of treating Overt Hepatic Encephalopathy (OHE) in a patient in need thereof comprising administering a rifaximin SSD IR composition (e.g., tablet) TID to the patient until the patient's Hepatic Encephalopathy Grading Instrument (HEGI) score is reduced by at least one grade, or at least two grades, or at least three grades.

In an embodiment, the invention described herein includes a method of treating Overt Hepatic Encephalopathy (OHE) in a patient in need thereof comprising administering a rifaximin SSD IR composition (e.g., tablet) TID to the patient until the patient's Hepatic Encephalopathy Grading Instrument (HEGI) score is reduced from grade 4 to grade 3 or from grade 3 to grade 2.

In an embodiment, the invention described herein includes a method of treating Overt Hepatic Encephalopathy (OHE) in a patient in need thereof comprising concomitantly administering lactulose and at least one rifaximin SSD IR composition (e.g., tablet) to the patient until the patient's Hepatic Encephalopathy Grading Instrument (HEGI) score is reduced by at least one grade, or at least two grades, or at least three grades.

In an embodiment, the invention described herein includes a method of treating Overt Hepatic Encephalopathy (OHE) in a patient in need thereof comprising administering lactulose and at least one rifaximin SSD IR composition (e.g., tablet) to the patient until the patient's Hepatic Encephalopathy Grading Instrument (HEGI) score is reduced from grade 4 to grade 3 or from grade 3 to grade 2.

In an embodiment, the invention described herein includes a method of treating Overt Hepatic Encephalopathy (OHE) in a patient in need thereof comprising administering lactulose and a rifaximin SSD IR composition (e.g., tablet) QD to the patient until the patient's Hepatic Encephalopathy Grading Instrument (HEGI) score is reduced by at least one grade, or at least two grades, or at least three grades.

In an embodiment, the invention described herein includes a method of treating Overt Hepatic Encephalopathy (OHE) in a patient in need thereof comprising administering lactulose and a rifaximin SSD IR composition (e.g., tablet) QD to the patient until the patient's Hepatic Encephalopathy Grading Instrument (REGI) score is reduced from grade 4 to grade 3 or from grade 3 to grade 2.

In an embodiment, the invention described herein includes a method of treating Overt Hepatic Encephalopathy (OHE) in a patient in need thereof comprising administering lactulose and a rifaximin SSD IR composition (e.g., tablet) BID to the patient until the patient's Hepatic Encephalopathy Grading Instrument (REGI) score is reduced by at least one grade, or at least two grades, or at least three grades.

In an embodiment, the invention described herein includes a method of treating Overt Hepatic Encephalopathy (OHE) in a patient in need thereof comprising administering lactulose and a rifaximin SSD IR composition (e.g., tablet) BID to the patient until the patient's Hepatic Encephalopathy Grading Instrument (REGI) score is reduced from grade 4 to grade 3 or from grade 3 to grade 2.

In an embodiment, the invention described herein includes a method of treating Overt Hepatic Encephalopathy (OHE) in a patient in need thereof comprising administering lactulose and a rifaximin SSD IR composition (e.g., tablet) TID to the patient until the patient's Hepatic Encephalopathy Grading Instrument (REGI) score is reduced by at least one grade, or at least two grades, or at least three grades.

In an embodiment, the invention described herein includes a method of treating Overt Hepatic Encephalopathy (OHE) in a patient in need thereof comprising administering lactulose and a rifaximin SSD IR composition (e.g., tablet) TID to the patient until the patient's Hepatic Encephalopathy Grading Instrument (REGI) score is reduced from grade 4 to grade 3 or from grade 3 to grade 2.

In an embodiment, the invention described herein includes a method of treating Overt Hepatic Encephalopathy (OHE) in a patient in need thereof comprising administering at least one rifaximin SSD IR composition (e.g., tablet) to the patient until the patient's West Haven Criteria (WHC) score is reduced by at least one grade, or at least two grades, or at least three grades.

In an embodiment, the invention described herein includes a method of treating Overt Hepatic Encephalopathy (OHE) in a patient in need thereof comprising administering at least one rifaximin SSD IR composition (e.g., tablet) to the patient until the patient's West Haven Criteria (WHC) score is reduced from grade 4 to grade 3 or from grade 3 to grade 2 or from grade 2 to grade 1 or from grade 1 to grade 0.

In an embodiment, the invention described herein includes a method of treating Overt Hepatic Encephalopathy (OHE) in a patient in need thereof comprising administering a rifaximin SSD IR composition (e.g., tablet) QD to the patient until the patient's West Haven Criteria (WHC) score is reduced by at least one grade, or at least two grades, or at least three grades.

In an embodiment, the invention described herein includes a method of treating Overt Hepatic Encephalopathy (OHE) in a patient in need thereof comprising administering a rifaximin SSD IR composition (e.g., tablet) QD to the patient until the patient's West Haven Criteria (WHC) score is reduced from grade 4 to grade 3 or from grade 3 to grade 2 or from grade 2 to grade 1 or from grade 1 to grade 0.

In an embodiment, the invention described herein includes a method of treating Overt Hepatic Encephalopathy (OHE) in a patient in need thereof comprising administering a rifaximin SSD IR composition (e.g., tablet) BID to the patient until the patient's West Haven Criteria (WHC) score is reduced by at least one grade, or at least two grades, or at least three grades.

In an embodiment, the invention described herein includes a method of treating Overt Hepatic Encephalopathy (OHE) in a patient in need thereof comprising administering a rifaximin SSD IR composition (e.g., tablet) BID to the patient until the patient's West Haven Criteria (WHC) score is reduced from grade 4 to grade 3 or from grade 3 to grade 2 or from grade 2 to grade 1 or from grade 1 to grade 0.

In an embodiment, the invention described herein includes a method of treating Overt Hepatic Encephalopathy (OHE) in a patient in need thereof comprising administering a rifaximin SSD IR composition (e.g., tablet) TID to the patient until the patient's West Haven Criteria (WHC) score is reduced by at least one grade, or at least two grades, or at least three grades.

In an embodiment, the invention described herein includes a method of treating Overt Hepatic Encephalopathy (OHE) in a patient in need thereof comprising administering a rifaximin SSD IR composition (e.g., tablet) TID to the patient until the patient's West Haven Criteria (WHC) score is reduced from grade 4 to grade 3 or from grade 3 to grade 2 or from grade 2 to grade 1 or from grade 1 to grade 0.

In an embodiment, the invention described herein includes a method of treating Overt Hepatic Encephalopathy (OHE) in a patient in need thereof comprising administering lactulose and at least one rifaximin SSD IR composition (e.g., tablet) to the patient until the patient's West Haven Criteria (WHC) score is reduced by at least one grade, or at least two grades, or at least three grades.

In an embodiment, the invention described herein includes a method of treating Overt Hepatic Encephalopathy (OHE) in a patient in need thereof comprising administering lactulose and at least one rifaximin SSD IR composition (e.g., tablet) to the patient until the patient's West Haven Criteria (WHC) score is reduced from grade 4 to grade 3 or from grade 3 to grade 2 or from grade 2 to grade 1 or from grade 1 to grade 0.

In an embodiment, the invention described herein includes a method of treating Overt Hepatic Encephalopathy (OHE) in a patient in need thereof comprising administering lactulose and a rifaximin SSD IR composition (e.g., tablet) QD to the patient until the patient's West Haven Criteria (WHC) score is reduced by at least one grade, or at least two grades, or at least three grades.

In an embodiment, the invention described herein includes a method of treating Overt Hepatic Encephalopathy (OHE) in a patient in need thereof comprising administering lactulose and a rifaximin SSD IR composition (e.g., tablet) QD to the patient until the patient's West Haven Criteria (WHC) score is reduced from grade 4 to grade 3 or from grade 3 to grade 2 or from grade 2 to grade 1 or from grade 1 to grade 0.

In an embodiment, the invention described herein includes a method of treating Overt Hepatic Encephalopathy (OHE) in a patient in need thereof comprising administering lactulose and a rifaximin SSD IR composition (e.g., tablet) BID to the patient until the patient's West Haven Criteria (WHC) score is reduced by at least one grade, or at least two grades, or at least three grades.

In an embodiment, the invention described herein includes a method of treating Overt Hepatic Encephalopathy (OHE) in a patient in need thereof comprising administering lactulose and a rifaximin SSD IR composition (e.g., tablet) BID to the patient until the patient's West Haven Criteria (WHC) score is reduced from grade 4 to grade 3 or from grade 3 to grade 2 or from grade 2 to grade 1 or from grade 1 to grade 0.

In an embodiment, the invention described herein includes a method of treating Overt Hepatic Encephalopathy (OHE) in a patient in need thereof comprising administering lactulose and a rifaximin SSD IR composition (e.g., tablet) TID to the patient until the patient's West Haven Criteria (WHC) score is reduced by at least one grade, or at least two grades, or at least three grades.

In an embodiment, the invention described herein includes a method of treating Overt Hepatic Encephalopathy (OHE) in a patient in need thereof comprising administering lactulose and a rifaximin SSD IR composition (e.g., tablet) TID to the patient until the patient's West Haven Criteria (WHC) score is reduced from grade 4 to grade 3 or from grade 3 to grade 2 or from grade 2 to grade 1 or from grade 1 to grade 0.

As described herein, it has been surprisingly discovered that rifaximin soluble solid dispersion (SSD) formulations were effective at preventing complications of liver cirrhosis or all-cause mortality, or both, in patients with liver decompensation. In an embodiment, an immediate release (IR) formulation of rifaximin as a soluble solid dispersion (SSD), having a 40 mg dose of rifaximin, was statistically more effective for a composite endpoint of all cause hospitalization/all-cause mortality (primary endpoint of the study was hospitalization for any of the liver cirrhosis complications (HE, EVB, SBP, or HRS).

For example, subjects in an alcohol-induced only subgroup had a statistically significant difference in time to all-cause hospitalization/all-cause Mortality versus placebo when treated with SSD IR 40 mg. Subjects with a Conn score of 0 demonstrated a statistically significant difference for both the overall treatment comparison of any rifaximin SSD treatment versus placebo and predominantly in favor of the IR 40 mg formulation. Results of a sensitivity analysis on the per protocol (PP) population demonstrated a statistically significant difference in the time to hospitalization for any of the liver cirrhosis complications or all-cause mortality up to 24 weeks that was in favor of an extended release (SER) formulation of rifaximin as a soluble solid dispersion (SSD), having a 80 mg dose of rifaximin versus placebo. Subjects in an alcohol-induced only subgroup had a statistically significant difference in time to all-cause hospitalization/all-cause mortality versus placebo when treated with rifaximin SER 80 mg QHS.

In an embodiment, the invention described herein includes a method of preventing complications of liver cirrhosis in a patient with liver decompensation comprising administering to the patient at least one (e.g., one) rifaximin SSD composition (e.g., tablet).

In an embodiment, the invention described herein includes a method of preventing complications of liver cirrhosis in a patient with liver decompensation comprising administering to the patient at least one (e.g., one) rifaximin SSD composition (e.g., tablet), wherein the patient has documented medically controlled non-refractory ascites.

In an embodiment, the invention described herein includes a method of preventing complications of liver cirrhosis in a patient with liver decompensation comprising administering to the patient at least one (e.g., one) rifaximin SSD composition (e.g., tablet), wherein the patient has not previously experienced spontaneous bacterial peritonitis (SBP), esophageal variceal bleeding (EVB), or hepatorenal syndrome (HRS) prior to treatment.

In an embodiment, the invention described herein includes a method of preventing complications of liver cirrhosis in a patient with liver decompensation comprising administering to the patient at least one (e.g., one) rifaximin SSD composition (e.g., tablet), wherein the patient has documented medically controlled non-refractory ascites and wherein the patient has not previously experienced spontaneous bacterial peritonitis (SBP), esophageal variceal bleeding (EVB), or hepatorenal syndrome (HRS) prior to treatment.

In an embodiment, the invention described herein includes a method of preventing complications of liver cirrhosis in a patient with liver decompensation comprising administering to the patient at least one (e.g., one) 40 mg rifaximin SSD IR composition (e.g., tablet).

In an embodiment, the invention described herein includes a method of preventing complications of liver cirrhosis in a patient with liver decompensation comprising administering to the patient at least one (e.g., one) 40 mg rifaximin SSD IR composition (e.g., tablet), wherein the patient has documented medically controlled non-refractory ascites.

In an embodiment, the invention described herein includes a method of preventing complications of liver cirrhosis in a patient with liver decompensation comprising administering to the patient at least one (e.g., one) 40 mg rifaximin SSD IR composition (e.g., tablet), wherein the patient has not previously experienced spontaneous bacterial peritonitis (SBP), esophageal variceal bleeding (EVB), or hepatorenal syndrome (HRS) prior to treatment.

In an embodiment, the invention described herein includes a method of preventing complications of liver cirrhosis in a patient with liver decompensation comprising administering to the patient at least one (e.g., one) 40 mg rifaximin SSD IR composition (e.g., tablet), wherein the patient has documented medically controlled non-refractory ascites and wherein the patient has not previously experienced spontaneous bacterial peritonitis (SBP), esophageal variceal bleeding (EVB), or hepatorenal syndrome (HRS) prior to treatment.

In an embodiment, the invention described herein includes a method of preventing complications of liver cirrhosis in a patient with liver decompensation comprising administering to the patient at least one (e.g., one) 80 mg rifaximin SSD SER composition (e.g., tablet).

In an embodiment, the invention described herein includes a method of preventing complications of liver cirrhosis in a patient with liver decompensation comprising administering to the patient at least one (e.g., one) 80 mg rifaximin SSD SER composition (e.g., tablet), wherein the patient has documented medically controlled non-refractory ascites.

In an embodiment, the invention described herein includes a method of preventing complications of liver cirrhosis in a patient with liver decompensation comprising administering to the patient at least one (e.g., one) 80 mg rifaximin SSD SER composition (e.g., tablet), wherein the patient has not previously experienced spontaneous bacterial peritonitis (SBP), esophageal variceal bleeding (EVB), or hepatorenal syndrome (HRS) prior to treatment.

In an embodiment, the invention described herein includes a method of preventing complications of liver cirrhosis in a patient with liver decompensation comprising administering to the patient at least one (e.g., one) 80 mg rifaximin SSD SER composition (e.g., tablet), wherein the patient has documented medically controlled non-refractory ascites and wherein the patient has not previously experienced spontaneous bacterial peritonitis (SBP), esophageal variceal bleeding (EVB), or hepatorenal syndrome (HRS) prior to treatment.

In an embodiment, the invention described herein includes a method of preventing all-cause mortality in patients with liver decompensation comprising administering to the patient at least one (e.g., one) rifaximin SSD composition (e.g., tablet).

In an embodiment, the invention described herein includes a method of preventing all-cause mortality in patients with liver decompensation comprising administering to the patient at least one (e.g., one) rifaximin SSD composition (e.g., tablet), wherein the patient has documented medically controlled non-refractory ascites.

In an embodiment, the invention described herein includes a method of preventing all-cause mortality in patients with liver decompensation comprising administering to the patient at least one (e.g., one) rifaximin SSD composition (e.g., tablet), wherein the patient has not previously experienced spontaneous bacterial peritonitis (SBP), esophageal variceal bleeding (EVB), or hepatorenal syndrome (HRS) prior to treatment.

In an embodiment, the invention described herein includes a method of preventing all-cause mortality in patients with liver decompensation comprising administering to the patient at least one (e.g., one) rifaximin SSD composition (e.g., tablet), wherein the patient has documented medically controlled non-refractory ascites and wherein the patient has not previously experienced spontaneous bacterial peritonitis (SBP), esophageal variceal bleeding (EVB), or hepatorenal syndrome (HRS) prior to treatment.

In an embodiment, the invention described herein includes a method of preventing all-cause mortality in patients with liver decompensation comprising administering to the patient at least one (e.g., one) 40 mg rifaximin SSD IR composition (e.g., tablet).

In an embodiment, the invention described herein includes a method of preventing all-cause mortality in patients with liver decompensation comprising administering to the patient at least one (e.g., one) 40 mg rifaximin SSD IR composition (e.g., tablet), wherein the patient has documented medically controlled non-refractory ascites.

In an embodiment, the invention described herein includes a method of preventing all-cause mortality in patients with liver decompensation comprising administering to the patient at least one (e.g., one) 40 mg rifaximin SSD IR composition (e.g., tablet), wherein the patient has not previously experienced spontaneous bacterial peritonitis (SBP), esophageal variceal bleeding (EVB), or hepatorenal syndrome (HRS) prior to treatment.

In an embodiment, the invention described herein includes a method of preventing all-cause mortality in patients with liver decompensation comprising administering to the patient at least one (e.g., one) 40 mg rifaximin SSD IR composition (e.g., tablet), wherein the patient has documented medically controlled non-refractory ascites and wherein the patient has not previously experienced spontaneous bacterial peritonitis (SBP), esophageal variceal bleeding (EVB), or hepatorenal syndrome (HRS) prior to treatment.

In an embodiment, the invention described herein includes a method of preventing all-cause mortality in patients with liver decompensation comprising administering to the patient at least one (e.g., one) 80 mg rifaximin SSD SER composition (e.g., tablet).

In an embodiment, the invention described herein includes a method of preventing all-cause mortality in patients with liver decompensation comprising administering to the patient at least one (e.g., one) 80 mg rifaximin SSD SER composition (e.g., tablet), wherein the patient has documented medically controlled non-refractory ascites.

In an embodiment, the invention described herein includes a method of preventing all-cause mortality in patients with liver decompensation comprising administering to the patient at least one (e.g., one) 80 mg rifaximin SSD SER composition (e.g., tablet), wherein the patient has not previously experienced spontaneous bacterial peritonitis (SBP), esophageal variceal bleeding (EVB), or hepatorenal syndrome (HRS) prior to treatment.

In an embodiment, the invention described herein includes a method of preventing all-cause mortality in patients with liver decompensation comprising administering to the patient at least one (e.g., one) 80 mg rifaximin SSD SER composition (e.g., tablet), wherein the patient has documented medically controlled non-refractory ascites and wherein the patient has not previously experienced spontaneous bacterial peritonitis (SBP), esophageal variceal bleeding (EVB), or hepatorenal syndrome (HRS) prior to treatment.

In an embodiment, the invention described herein includes a method of preventing complications of liver cirrhosis and all-cause mortality in patients with liver decompensation comprising administering to the patient at least one (e.g., one) rifaximin SSD composition (e.g., tablet).

In an embodiment, the invention described herein includes a method of preventing complications of liver cirrhosis and all-cause mortality in patients with liver decompensation comprising administering to the patient at least one (e.g., one) rifaximin SSD composition (e.g., tablet), wherein the patient has documented medically controlled non-refractory ascites.

In an embodiment, the invention described herein includes a method of preventing complications of liver cirrhosis and all-cause mortality in patients with liver decompensation comprising administering to the patient at least one (e.g., one) rifaximin SSD composition (e.g., tablet), wherein the patient has not previously experienced spontaneous bacterial peritonitis (SBP), esophageal variceal bleeding (EVB), or hepatorenal syndrome (HRS) prior to treatment.

In an embodiment, the invention described herein includes a method of preventing complications of liver cirrhosis and all-cause mortality in patients with liver decompensation comprising administering to the patient at least one (e.g., one) rifaximin SSD composition (e.g., tablet), wherein the patient has documented medically controlled non-refractory ascites and wherein the patient has not previously experienced spontaneous bacterial peritonitis (SBP), esophageal variceal bleeding (EVB), or hepatorenal syndrome (HRS) prior to treatment.

In an embodiment, the invention described herein includes a method of preventing complications of liver cirrhosis and all-cause mortality in patients with liver decompensation comprising administering to the patient at least one (e.g., one) 40 mg rifaximin SSD IR composition (e.g., tablet).

In an embodiment, the invention described herein includes a method of preventing complications of liver cirrhosis and all-cause mortality in patients with liver decompensation comprising administering to the patient at least one (e.g., one) 40 mg rifaximin SSD IR composition (e.g., tablet), wherein the patient has documented medically controlled non-refractory ascites.

In an embodiment, the invention described herein includes a method of preventing complications of liver cirrhosis and all-cause mortality in patients with liver decompensation comprising administering to the patient at least one (e.g., one) 40 mg rifaximin SSD IR composition (e.g., tablet), wherein the patient has not previously experienced spontaneous bacterial peritonitis (SBP), esophageal variceal bleeding (EVB), or hepatorenal syndrome (HRS) prior to treatment.

In an embodiment, the invention described herein includes a method of preventing complications of liver cirrhosis and all-cause mortality in patients with liver decompensation comprising administering to the patient at least one (e.g., one) 40 mg rifaximin SSD IR composition (e.g., tablet), wherein the patient has not previously experienced spontaneous bacterial peritonitis (SBP), hepatic encephalopathy (HE), esophageal variceal bleeding (EVB), or hepatorenal syndrome (HRS) prior to treatment.

In an embodiment, the invention described herein includes a method of preventing complications of liver cirrhosis and all-cause mortality in patients with liver decompensation comprising administering to the patient at least one (e.g., one) 40 mg rifaximin SSD IR composition (e.g., tablet), wherein the patient has documented medically controlled non-refractory ascites and wherein the patient has not previously experienced spontaneous bacterial peritonitis (SBP), esophageal variceal bleeding (EVB), or hepatorenal syndrome (HRS) prior to treatment.

In an embodiment, the invention described herein includes a method of preventing complications of liver cirrhosis and all-cause mortality in patients with liver decompensation comprising administering to the patient at least one (e.g., one) 40 mg rifaximin SSD IR composition (e.g., tablet), wherein the patient has documented medically controlled non-refractory ascites and wherein the patient has not previously experienced spontaneous bacterial peritonitis (SBP), hepatic encephalopathy (HE), esophageal variceal bleeding (EVB), or hepatorenal syndrome (HRS) prior to treatment.

In an embodiment, the invention described herein includes a method of preventing complications of liver cirrhosis and all-cause mortality in patients with liver decompensation comprising administering to the patient at least one (e.g., one) 80 mg rifaximin SSD SER composition (e.g., tablet).

In an embodiment, the invention described herein includes a method of preventing complications of liver cirrhosis and all-cause mortality in patients with liver decompensation comprising administering to the patient at least one (e.g., one) 80 mg rifaximin SSD SER composition (e.g., tablet), wherein the patient has documented medically controlled non-refractory ascites.

In an embodiment, the invention described herein includes a method of preventing complications of liver cirrhosis and all-cause mortality in patients with liver decompensation comprising administering to the patient at least one (e.g., one) 80 mg rifaximin SSD SER composition (e.g., tablet), wherein the patient has not previously experienced spontaneous bacterial peritonitis (SBP), esophageal variceal bleeding (EVB), or hepatorenal syndrome (HRS) prior to treatment.

In an embodiment, the invention described herein includes a method of preventing complications of liver cirrhosis and all-cause mortality in patients with liver decompensation comprising administering to the patient at least one (e.g., one) 80 mg rifaximin SSD SER composition (e.g., tablet), wherein the patient has not previously experienced spontaneous bacterial peritonitis (SBP), hepatic encephalopathy (HE), esophageal variceal bleeding (EVB), or hepatorenal syndrome (HRS) prior to treatment.

In an embodiment, the invention described herein includes a method of preventing complications of liver cirrhosis and all-cause mortality in patients with liver decompensation comprising administering to the patient at least one (e.g., one) 80 mg rifaximin SSD SER composition (e.g., tablet), wherein the patient has documented medically controlled non-refractory ascites and wherein the patient has not previously experienced spontaneous bacterial peritonitis (SBP), esophageal variceal bleeding (EVB), or hepatorenal syndrome (HRS) prior to treatment.

In an embodiment, the invention described herein includes a method of preventing complications of liver cirrhosis and all-cause mortality in patients with liver decompensation comprising administering to the patient at least one (e.g., one) 80 mg rifaximin SSD SER composition (e.g., tablet), wherein the patient has documented medically controlled non-refractory ascites and wherein the patient has not previously experienced spontaneous bacterial peritonitis (SBP), esophageal variceal bleeding (EVB), hepatic encephalopathy (HE), or hepatorenal syndrome (HRS) prior to treatment.

In an embodiment, the invention described herein includes a method of preventing hospitalizations associated with complications of liver cirrhosis in patients with liver decompensation comprising administering to the patient at least one (e.g., one) rifaximin SSD composition (e.g., tablet).

In an embodiment, the invention described herein includes a method of preventing hospitalizations associated with complications of liver cirrhosis in patients with liver decompensation comprising administering to the patient at least one (e.g., one) rifaximin SSD composition (e.g., tablet), wherein the patient has documented medically controlled non-refractory ascites.

In an embodiment, the invention described herein includes a method of preventing hospitalizations associated with complications of liver cirrhosis in patients with liver decompensation comprising administering to the patient at least one (e.g., one) rifaximin SSD composition (e.g., tablet), wherein the patient has not previously experienced spontaneous bacterial peritonitis (SBP), esophageal variceal bleeding (EVB), or hepatorenal syndrome (HRS) prior to treatment.

In an embodiment, the invention described herein includes a method of preventing hospitalizations associated with complications of liver cirrhosis in patients with liver decompensation comprising administering to the patient at least one (e.g., one) rifaximin SSD composition (e.g., tablet), wherein the patient has not previously experienced spontaneous bacterial peritonitis (SBP), esophageal variceal bleeding (EVB), hepatic encephalopathy (HE), or hepatorenal syndrome (HRS) prior to treatment.

In an embodiment, the invention described herein includes a method of preventing hospitalizations associated with complications of liver cirrhosis in patients with liver decompensation comprising administering to the patient at least one (e.g., one) rifaximin SSD composition (e.g., tablet), wherein the patient has documented medically controlled non-refractory ascites and wherein the patient has not previously experienced spontaneous bacterial peritonitis (SBP), esophageal variceal bleeding (EVB), or hepatorenal syndrome (HRS) prior to treatment.

In an embodiment, the invention described herein includes a method of preventing hospitalizations associated with complications of liver cirrhosis in patients with liver decompensation comprising administering to the patient at least one (e.g., one) rifaximin SSD composition (e.g., tablet), wherein the patient has documented medically controlled non-refractory ascites and wherein the patient has not previously experienced spontaneous bacterial peritonitis (SBP), esophageal variceal bleeding (EVB), hepatic encephalopathy (HE), or hepatorenal syndrome (HRS) prior to treatment.

In an embodiment, the invention described herein includes a method of preventing hospitalizations associated with complications of liver cirrhosis in patients with liver decompensation comprising administering to the patient at least one (e.g., one) 40 mg rifaximin SSD IR composition (e.g., tablet).

In an embodiment, the invention described herein includes a method of preventing hospitalizations associated with complications of liver cirrhosis in patients with liver decompensation comprising administering to the patient at least one (e.g., one) 40 mg rifaximin SSD IR composition (e.g., tablet), wherein the patient has documented medically controlled non-refractory ascites.

In an embodiment, the invention described herein includes a method of preventing hospitalizations associated with complications of liver cirrhosis in patients with liver decompensation comprising administering to the patient at least one (e.g., one) 40 mg rifaximin SSD IR composition (e.g., tablet), wherein the patient has not previously experienced spontaneous bacterial peritonitis (SBP), esophageal variceal bleeding (EVB), or hepatorenal syndrome (HRS) prior to treatment.

In an embodiment, the invention described herein includes a method of preventing hospitalizations associated with complications of liver cirrhosis in patients with liver decompensation comprising administering to the patient at least one (e.g., one) 40 mg rifaximin SSD IR composition (e.g., tablet), wherein the patient has not previously experienced spontaneous bacterial peritonitis (SBP), esophageal variceal bleeding (EVB), hepatic encephalopathy (HE), or hepatorenal syndrome (HRS) prior to treatment.

In an embodiment, the invention described herein includes a method of preventing hospitalizations associated with complications of liver cirrhosis in patients with liver decompensation comprising administering to the patient at least one (e.g., one) 40 mg rifaximin SSD IR composition (e.g., tablet), wherein the patient has documented medically controlled non-refractory ascites and wherein the patient has not previously experienced spontaneous bacterial peritonitis (SBP), esophageal variceal bleeding (EVB), or hepatorenal syndrome (HRS) prior to treatment.

In an embodiment, the invention described herein includes a method of preventing hospitalizations associated with complications of liver cirrhosis in patients with liver decompensation comprising administering to the patient at least one (e.g., one) 40 mg rifaximin SSD IR composition (e.g., tablet), wherein the patient has documented medically controlled non-refractory ascites and wherein the patient has not previously experienced spontaneous bacterial peritonitis (SBP), esophageal variceal bleeding (EVB), hepatic encephalopathy (HE), or hepatorenal syndrome (HRS) prior to treatment.

In an embodiment, the invention described herein includes a method of preventing hospitalizations associated with complications of liver cirrhosis in patients with liver decompensation comprising administering to the patient at least one (e.g., one) 80 mg rifaximin SSD SER composition (e.g., tablet).

In an embodiment, the invention described herein includes a method of preventing hospitalizations associated with complications of liver cirrhosis in patients with liver decompensation comprising administering to the patient at least one (e.g., one) 80 mg rifaximin SSD SER composition (e.g., tablet), wherein the patient has documented medically controlled non-refractory ascites.

In an embodiment, the invention described herein includes a method of preventing hospitalizations associated with complications of liver cirrhosis in patients with liver decompensation comprising administering to the patient at least one (e.g., one) 80 mg rifaximin SSD SER composition (e.g., tablet), wherein the patient has not previously experienced spontaneous bacterial peritonitis (SBP), esophageal variceal bleeding (EVB), or hepatorenal syndrome (HRS) prior to treatment.

In an embodiment, the invention described herein includes a method of preventing hospitalizations associated with complications of liver cirrhosis in patients with liver decompensation comprising administering to the patient at least one (e.g., one) 80 mg rifaximin SSD SER composition (e.g., tablet), wherein the patient has not previously experienced spontaneous bacterial peritonitis (SBP), esophageal variceal bleeding (EVB), hepatic encephalopathy (HE), or hepatorenal syndrome (HRS) prior to treatment.

In an embodiment, the invention described herein includes a method of preventing hospitalizations associated with complications of liver cirrhosis in patients with liver decompensation comprising administering to the patient at least one (e.g., one) 80 mg rifaximin SSD SER composition (e.g., tablet), wherein the patient has documented medically controlled non-refractory ascites and wherein the patient has not previously experienced spontaneous bacterial peritonitis (SBP), esophageal variceal bleeding (EVB), or hepatorenal syndrome (HRS) prior to treatment.

In an embodiment, the invention described herein includes a method of preventing hospitalizations associated with complications of liver cirrhosis in patients with liver decompensation comprising administering to the patient at least one (e.g., one) 80 mg rifaximin SSD SER composition (e.g., tablet), wherein the patient has documented medically controlled non-refractory ascites and wherein the patient has not previously experienced spontaneous bacterial peritonitis (SBP), esophageal variceal bleeding (EVB), hepatic encephalopathy (HE), or hepatorenal syndrome (HRS) prior to treatment.

In the preceding embodiments relating to preventing complications of liver cirrhosis or all-cause mortality in patients, or both, in patients with liver decompensation, the rifaximin SSD composition may be administered to the patients for at least one day, or at least two days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days.

In the preceding embodiments relating to preventing complications of liver cirrhosis or all-cause mortality in patients, or both, in patients with liver decompensation, the rifaximin SSD composition may be administered to the patients once, twice, or three times a day.

In the embodiments relating to preventing complications of liver cirrhosis or all-cause mortality in patients, or both, in patients with liver decompensation, the rifaximin SSD composition may be administered to patients at a dose of 40 mg (e.g., one rifaximin SSD 40 mg IR tablet), 80 mg (e.g., two rifaximin SSD 40 mg IR tablets), or 120 mg (e.g., three rifaximin SSD 40 mg IR tablets).

In the preceding embodiments relating to preventing complications of liver cirrhosis or all-cause mortality in patients, or both, in patients with liver decompensation, the rifaximin SSD composition may be administered to the patients once, twice, or three times a day for a period of time of at least one day, or at least two days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days.

In the preceding embodiments relating to preventing hospitalizations associated with complications of liver cirrhosis in patients with liver decompensation, the rifaximin SSD composition may be administered to the patients for at least one day, or at least two days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days.

In the preceding embodiments relating to preventing hospitalizations associated with complications of liver cirrhosis in patients with liver decompensation, the rifaximin SSD composition may be administered to the patients once, twice, or three times a day.

In the embodiments relating to preventing hospitalizations associated with complications of liver cirrhosis in patients with liver decompensation, the rifaximin SSD composition may be administered to patients at a dose of 40 mg (e.g., one rifaximin SSD 40 mg IR tablet), 80 mg (e.g., two rifaximin SSD 40 mg IR tablets), or 120 mg (e.g., three rifaximin SSD 40 mg IR tablets).

In the preceding embodiments relating to preventing hospitalizations associated with complications of liver cirrhosis in patients with liver decompensation, the rifaximin SSD composition may be administered to the patients once, twice, or three times a day for a period of time of at least one day, or at least two days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days.

In an embodiment, the invention described herein includes a method of delaying the onset of overt hepatic encephalopathy (OHE) in patients with liver decompensation comprising administering to the patient at least one (e.g., one) rifaximin SSD composition (e.g., tablet).

In an embodiment, the invention described herein includes a method of delaying the onset of overt hepatic encephalopathy (OHE) in patients with liver decompensation comprising administering to the patient at least one (e.g., one) 40 mg rifaximin SSD IR. composition (e.g., tablet).

In an embodiment, the invention described herein includes a method of delaying the onset of overt hepatic encephalopathy (OHE) in patients with liver decompensation comprising administering to the patient at least one (e.g., one) rifaximin SSD composition (e.g., tablet), wherein liver decompensation comprises cirrhosis of the liver and medically controlled ascites.

In an embodiment, the invention described herein includes a method of delaying the onset of overt hepatic encephalopathy (OHE) in patients with liver decompensation comprising administering to the patient at least one (e.g., one) 40 mg rifaximin SSD IR. composition (e.g., tablet), wherein liver decompensation comprises cirrhosis of the liver and medically controlled ascites.

In an embodiment, the invention described herein includes a method of delaying the onset of overt hepatic encephalopathy (OHE) in patients with liver decompensation comprising administering to the patient at least one (e.g., one) rifaximin SSD composition (e.g., tablet), wherein liver decompensation comprises cirrhosis of the liver and medically controlled ascites for a period of at least 30 days.

In an embodiment, the invention described herein includes a method of delaying the onset of overt hepatic encephalopathy (OHE) in patients with liver decompensation comprising administering to the patient at least one (e.g., one) 40 mg rifaximin SSD IR. composition (e.g., tablet), wherein liver decompensation comprises cirrhosis of the liver and medically controlled ascites for a period of at least 30 days.

In an embodiment, the invention described herein includes a method of delaying the onset of overt hepatic encephalopathy (OHE) in patients with liver decompensation comprising administering to the patient at least one (e.g., one) rifaximin SSD composition (e.g., tablet), wherein the patient has one or more of a Child-Pugh B Classification score of from 7 to 9 without OHE prior to treatment, a Model End Stage Liver Disease-Sodium (MELD-Na) score of less than 15 prior to treatment, a Conn (West Haven Criteria) score of less than 2 prior to treatment, and a Mini-Mental State Examination (MMSE) score of at least 25 prior to treatment. In an embodiment, the patient has a Child-Pugh B Classification score of from 7 to 9 without OHE prior to treatment. In an embodiment, the patient has a Model End Stage Liver Disease-Sodium (MELD-Na) score of less than 15 prior to treatment. In an embodiment, the patient has a Conn (West Haven Criteria) score of less than 2 prior to treatment. In an embodiment, the patient has a Mini-Mental State Examination (MMSE) score of at least 25 prior to treatment.

In an embodiment, the invention described herein includes a method of delaying the onset of overt hepatic encephalopathy (OHE) in patients with liver decompensation comprising administering to the patient at least one (e.g., one) 40 mg rifaximin SSD IR. composition (e.g., tablet), wherein the patient has one or more of a Child-Pugh B Classification score of from 7 to 9 without OHE prior to treatment, a Model End Stage Liver Disease-Sodium (MELD-Na) score of less than 15 prior to treatment, a Conn (West Haven Criteria) score of less than 2 prior to treatment, and a Mini-Mental State Examination (MMSE) score of at least 25 prior to treatment. In an embodiment, the patient has a Child-Pugh B Classification score of from 7 to 9 without OHE prior to treatment. In an embodiment, the patient has a Model End Stage Liver Disease-Sodium (MELD-Na) score of less than 15 prior to treatment. In an embodiment, the patient has a Conn (West Haven Criteria) score of less than 2 prior to treatment. In an embodiment, the patient has a Mini-Mental State Examination (MMSE) score of at least 25 prior to treatment.

In an embodiment, the invention described herein includes a method of delaying the onset of overt hepatic encephalopathy (OHE) in patients with liver decompensation comprising administering to the patient at least one (e.g., one) rifaximin SSD composition (e.g., tablet), wherein liver decompensation comprises cirrhosis of the liver and medically controlled ascites, and wherein the patient has one or more of a Child-Pugh B Classification score of from 7 to 9 without OHE prior to treatment, a Model End Stage Liver Disease-Sodium (MELD-Na) score of less than 15 prior to treatment, a Conn (West Haven Criteria) score of less than 2 prior to treatment, and a Mini-Mental State Examination (MMSE) score of at least 25 prior to treatment. In an embodiment, the patient has a Child-Pugh B Classification score of from 7 to 9 without OHE prior to treatment. In an embodiment, the patient has a Model End Stage Liver Disease-Sodium (MELD-Na) score of less than 15 prior to treatment. In an embodiment, the patient has a Conn (West Haven Criteria) score of less than 2 prior to treatment. In an embodiment, the patient has a Mini-Mental State Examination (MMSE) score of at least 25 prior to treatment.

In an embodiment, the invention described herein includes a method of delaying the onset of overt hepatic encephalopathy (OHE) in patients with liver decompensation comprising administering to the patient at least one (e.g., one) 40 mg rifaximin SSD IR. composition (e.g., tablet), wherein liver decompensation comprises cirrhosis of the liver and medically controlled ascites, and wherein the patient has one or more of a Child-Pugh B Classification score of from 7 to 9 without OHE prior to treatment, a Model End Stage Liver Disease-Sodium (MELD-Na) score of less than 15 prior to treatment, a Conn (West Haven Criteria) score of less than 2 prior to treatment, and a Mini-Mental State Examination (MMSE) score of at least 25 prior to treatment. In an embodiment, the patient has a Child-Pugh B Classification score of from 7 to 9 without OHE prior to treatment. In an embodiment, the patient has a Model End Stage Liver Disease-Sodium (MELD-Na) score of less than 15 prior to treatment. In an embodiment, the patient has a Conn (West Haven Criteria) score of less than 2 prior to treatment. In an embodiment, the patient has a Mini-Mental State Examination (MMSE) score of at least 25 prior to treatment.

In an embodiment, the invention described herein includes a method of delaying the onset of overt hepatic encephalopathy (OHE) in patients with liver decompensation comprising administering to the patient at least one (e.g., one) rifaximin SSD composition (e.g., tablet), wherein liver decompensation comprises cirrhosis of the liver and medically controlled ascites for a period of at least 30 days, and wherein the patient has one or more of a Child-Pugh B Classification score of from 7 to 9 without OHE prior to treatment, a Model End Stage Liver Disease-Sodium (MELD-Na) score of less than 15 prior to treatment, a Conn (West Haven Criteria) score of less than 2 prior to treatment, and a Mini-Mental State Examination (MMSE) score of at least 25 prior to treatment. In an embodiment, the patient has a Child-Pugh B Classification score of from 7 to 9 without OHE prior to treatment. In an embodiment, the patient has a Model End Stage Liver Disease-Sodium (MELD-Na) score of less than 15 prior to treatment. In an embodiment, the patient has a Conn (West Haven Criteria) score of less than 2 prior to treatment. In an embodiment, the patient has a Mini-Mental State Examination (MMSE) score of at least 25 prior to treatment.

In an embodiment, the invention described herein includes a method of delaying the onset of overt hepatic encephalopathy (OHE) in patients with liver decompensation comprising administering to the patient at least one (e.g., one) 40 mg rifaximin SSD IR. composition (e.g., tablet), wherein liver decompensation comprises cirrhosis of the liver and medically controlled ascites for a period of at least 30 days, and wherein the patient has one or more of a Child-Pugh B Classification score of from 7 to 9 without OHE prior to treatment, a Model End Stage Liver Disease-Sodium (MELD-Na) score of less than 15 prior to treatment, a Conn (West Haven Criteria) score of less than 2 prior to treatment, and a Mini-Mental State Examination (MMSE) score of at least 25 prior to treatment. In an embodiment, the patient has a Child-Pugh B Classification score of from 7 to 9 without OHE prior to treatment. In an embodiment, the patient has a Model End Stage Liver Disease-Sodium (MELD-Na) score of less than 15 prior to treatment. In an embodiment, the patient has a Conn (West Haven Criteria) score of less than 2 prior to treatment. In an embodiment, the patient has a Mini-Mental State Examination (MMSE) score of at least 25 prior to treatment.

In the preceding embodiments relating to delaying the onset of overt hepatic encephalopathy (OHE) in patients with liver decompensation, the patient demonstrates no cognitive impairment prior to treatment.

In the preceding embodiments relating to delaying the onset of overt hepatic encephalopathy (OHE) in patients with liver decompensation, the patient does not require paracentesis.

In the preceding embodiments relating to delaying the onset of overt hepatic encephalopathy (OHE) in patients with liver decompensation, the patient demonstrates no cognitive impairment prior to treatment and does not require paracentesis.

In the preceding embodiments relating to delaying the onset of overt hepatic encephalopathy (OHE) in patients with liver decompensation, the rifaximin SSD composition may be administered to the patients for at least one day, or at least two days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days.

In the preceding embodiments relating to delaying the onset of overt hepatic encephalopathy (OHE) in patients with liver decompensation, the rifaximin SSD composition may be administered to the patients once, twice, or three times a day.

In the embodiments relating to delaying the onset of overt hepatic encephalopathy (OHE) in patients with liver decompensation, the rifaximin SSD composition may be administered to patients at a dose of 40 mg (e.g., one rifaximin SSD 40 mg IR tablet), 80 mg (e.g., two rifaximin SSD 40 mg IR tablets), or 120 mg (e.g., three rifaximin SSD 40 mg IR tablets).

In the preceding embodiments relating to delaying the onset of overt hepatic encephalopathy (OHE) in patients with liver decompensation, the rifaximin SSD composition may be administered once, twice, or three times a day to the patients for a period of time of at least one day, or at least two days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days.

In the embodiments described herein, where rifaximin is administered with lactulose, the rifaximin and lactulose may be administered to the patient concomitantly or sequentially.

### BRIEF DESCRIPTION OF THE FIGURES

FIG. 1 presents a Kaplan-Meier estimate for the distribution of time to hospitalization for any of the liver cirrhosis complications by treatment group for an ITT (intent-to-treat) population with a formulation comprising rifaximin solid dispersion.

FIG. 2 presents a Kaplan-Meier estimate for the distribution of time to all-cause mortality by treatment group for an ITT population with a formulation comprising rifaximin solid dispersion.

FIG. 3 presents a Kaplan-Meier estimate for the distribution of time to hospitalization for any of the liver cirrhosis complications or all-cause mortality by treatment group for the ITT population with a formulation comprising rifaximin solid dispersion.

FIG. 4 presents a Kaplan-Meier estimate for the distribution of time to hospitalization for any of the liver cirrhosis complications or all-cause mortality by treatment group for the PP (per protocol) population with a formulation comprising rifaximin solid dispersion.

FIG. 5 presents a table demonstrating time to overt hepatic encephalopathy (OHE) resolution by Hepatic Encephalopathy Grading Instrument (HEGI).

FIG. 6 presents a table demonstrating treatment-emergent adverse events in an OHE study.

FIG. 7 present the study design for evaluating the efficacy and safety of rifaximin SSD-40IR for the delay of early encephalopathy decompensation in subjects with advanced liver cirrhosis

### DETAILED DESCRIPTION

Provided herein are solid dispersions comprising rifaximin and hydroxypropyl methylcellulose acetate succinate (HPMC-AS) and methods of using the same for the treatment of Overt Hepatic Encephalopathy (OHE).

### Definitions

The term “solid dispersion” or as used herein refers to a dispersion of rifaximin and an inert carrier matrix in a solid form, i.e., rifaximin is homologously mixed with an inert carrier. The inert matrix is generally hydrophilic (e.g., a polymer such as HPMC-AS) and may be crystalline or amorphous. It will be understood that it is not necessarily the preparation method that governs the properties of the solid dispersion, but rather the molecular arrangement of the contents of the dispersion. Thus, absent an expression to do so, or an incorporation of process restrictions, solid dispersions are not to be limited by the process to which they are made. The terms “solid dispersion”, “soluble solid dispersion”, and the abbreviations “SD” or “SSD” are used interchangeably and each refer to the disclosed solid dispersion of rifaximin.

As used herein the terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

The language “therapeutically effective amount” refers to an amount of a composition comprising a solid dispersion of rifaximin effective, upon single or multiple dose administration to the subject to provide a therapeutic benefit to the subject.

“Hepatic encephalopathy” or “HE” for shorthand is defined as an altered mental status diagnosed as HE and defined as an increase of the Conn score to Grade ≥2 (i.e., 0 or 1 to ≥2). HE may be considered “covert” or “overt” HE (CHE or OHE, respectively) depending upon the severity of the symptoms associated therewith. HE may be described as a continuum denoted by the West Haven Criteria (WHC): Grade 0—Minimal hepatic encephalopathy with symptoms potentially including impaired complex and sustained attention, or no personality or behavioral abnormality detected; Grade 1 (CHE)—Symptoms include trivial lack of awareness, euphoria or anxiety, shortened attention span, impairment of addition or subtraction, and altered sleep rhythm where clinical findings include mild asterixis or tremor; Grade 2 (OHE)—Symptoms include lethargy or apathy, disorientation for time, obvious personality change, and inappropriate behavior where clinical findings include obvious asterixis, dyspraxia, and slurred speech; Grade 3 (OHE)—Symptoms include somnolence to semistupor, responsive to stimuli, confused, gross disorientation, and bizarre behavior where clinical findings include muscular rigidity, clonus, and hyperreflexia; and Grade 4 (OHE)—Symptoms include coma where clinical findings include decerebrate posturing. OHE may also be observed on the Hepatic Encephalopathy Grading Instrument (HEGI), which uses clinical findings (present for at least 1 hour) to measure a patient's disorientation and thereby the severity of an HE episode (on a scale of Grade 2 to Grade 4)—Grade 4 being the most severe and Grade 2 being the least severe. In one aspect, OHE as referred to in the methods described herein means a Conn score of 2 or greater, e.g., Grade 2, Grade 3, or Grade 4.

“Esophageal variceal bleeding” or “EVB” for shorthand is defined as the occurrence of a clinically significant gastrointestinal bleed being defined as 1) bleeding from an esophageal or gastric varix at the time of endoscopy or 2) the presence of large varices with blood evident in the stomach, and no other identifiable cause of bleeding observed during endoscopy, and at least one or more of the following criteria is present: i) drop in hemoglobin of greater than 2 g/dL over the first 48 hours post hospital admission, ii) transfusion requirement of 2 units of blood or more within 24 hours of hospital admission, iii) a systolic blood pressure of less than 100 mm Hg, or iv) pulse rate greater than 100 beat/min at the time of admission.

“Spontaneous bacterial peritonitis or “SBP” for shorthand is defined as greater than 250 polymorphonuclear (PMN) cells/mm³ and/or positive monomicrobial culture in the ascitic fluid.

“Hepatorenal syndrome” (HRS) is defined as i) progressive rise in serum creatinine (>1.5 mg/dL) with no improvement after at least 2 days with diuretic withdrawal and volume expansion with albumin, ii) absence of parenchymal kidney disease, iii) oliguria, iv) absence of shock, and v) no current or recent (within 3 months prior randomization) treatment with nephrotoxic drugs.

“Time to development of medically refractory ascites” is defined as ascites which can either no longer be effectively managed by i) a low sodium diet and maximal doses of diuretics (e.g., up to 400 mg spironolactone and 160 mg furosemide per day) or ii) diuretics, due to the inability to tolerate side effects of maximal doses of diuretics.

For the purposes of this application, “cirrhosis of the liver” or “cirrhosis” refers to the condition in which a subject's liver is scarred and permanently damaged. Cirrhosis of the liver may be diagnosed according to methods known in the art and/or by medical professionals. In one aspect, cirrhosis may be determined by one or more of histopathological evidence of cirrhosis, transient elastography, the presence of esophageal varices, and thrombocytopenia characterized by a blood platelet level of less than 150,000 in a patient with chronic liver disease. The severity of cirrhosis may also be characterized by methods known in the art and/or by medical professionals. In one aspect, the severity of cirrhosis is characterized by Child-Pugh score.

The Child-Pugh scoring system (also known as the Child-Pugh-Turcotte score) refers to the Child-Pugh classification system for the severity of liver disease is listed below. The classification system determines severity of liver disease according to the degree of ascites (via clinical assessment), the serum concentrations of bilirubin and albumin, International Normalized Ratio (INR), and the degree of encephalopathy. Encephalopathy is classified as Grade 0-4 using the Conn scoring system provided above. A total score of 5-6 is considered grade A (well-compensated disease); 7-9 is grade B (significant functional compromise); and 10-15 is grade C (decompensated disease).

| | Points Assigned | | |
|---|---|---|---|
| Parameter | 1 | 2 | 3 |
| Ascites | Absent | Mild | Moderate to |
| | | | Severe |
| Bilirubin, mg/dL | <2 | 2-3 | >3 |
| Albumin, g/dL | >3.5 | 2.8-3.5 | <2.8 |
| International | <1.7 | 1.71-2.20 | >2.20 |
| Normalized Ratio | | | |
| Encephalopathy | None | Grade 1-2 | Grade 3-4 |

Complications of cirrhosis refer to one or more medical problems arising from the underlying disease, cirrhosis. In one aspect, complications include varices, splenomegaly, jaundice, esophageal variceal bleeding (EVB), portal hypertension. ascites, edema, hepatic encephalopathy (HE), hepatopulmonary hypertension, hepatocellular carcinoma, hepatorenal syndrome (HRS), spontaneous bacterial peritonitis (SBP), and coagulation disorders. In one aspect, complications of cirrhosis include one or more of hepatic encephalopathy (HE), esophageal variceal bleeding (EVB), spontaneous bacterial peritonitis (SBP), and hepatorenal syndrome (HRS).

The model for end stage liver disease (MELD) score refers to a scoring system used in assessing the severity of chronic liver disease. As used herein, a MELD score is calculated using the following formula: (0.957*ln(Serum Cr mg/dL)+0.378*ln(Serum Bilirubin mg/dL)+1.120*ln(INR)+0.643)*10. MELD-Na score will be calculated using the following formula: MELD−Na−[0.025×MELD×(140−Na)]+140.

The mini-mental state examination (MMSE) is known in the art and refers to a brief screening test that quantitatively assesses the severity of cognitive impairment and documents cognitive changes occurring over time. It includes 11 questions that test cognitive function, orientation, attention, memory (immediate and short-term recall), language, and the ability to follow simple verbal and written commands. See e.g., Cullum et al., Mini-Mental State Examination (MMSE) for the detection of Alzheimer's disease and other dementias in people with mild cognitive impairment (MCI). Cochrane Database of Systematic Reviews 2015, Issue 3. Art. No.: CD010783. DOI: 10.1002/14651858.CD010783.pub2. Decompensated cirrhosis or decompensated liver cirrhosis is an advanced state of cirrhosis and generally refers to an acute deterioration in liver function in a patient with cirrhosis. Decompensated cirrhosis may be characterized by one or more of jaundice, ascites, hepatic encephalopathy, hepatorenal syndrome or variceal haemorrhage. In one aspect, early decompensated liver cirrhosis refers to subjects who are diagnosed with advanced liver cirrhosis and have medically controlled ascites (>30 days) not requiring paracentesis (prophylactic variceal banding allowed if no history of previous variceal bleeding).

The terms “treatment,” “treat,” and “treating” refer to reversing, alleviating, reducing the likelihood of developing, or inhibiting the progress of a disease or disorder, or one or more symptoms thereof, as described herein. In some embodiments, treatment may be administered after one or more symptoms have developed, i.e., therapeutic treatment. In other embodiments, treatment may be administered in the absence of symptoms. For example, treatment may be administered to a susceptible individual prior to the onset of symptoms (e.g., in light of a history of symptoms and/or in light of genetic or other susceptibility factors), i.e., prophylactic treatment to prevent occurrence of a disease or disorder, or one or more symptoms thereof, as described herein. Treatment may also be continued after symptoms have resolved, for example to prevent or delay their recurrence.

As used herein, when a numerical value is modified by the term “about”, the exact numerical value is also deemed to be disclosed.

### Compositions

In a first embodiment, the present disclosure provides a solid dispersion comprising rifaximin and HPMC-AS.

In a second embodiment, the present disclosure provides a solid dispersion comprising rifaximin and HPMC-AS, wherein the HPMC-AS is present in an amount of from about 10 wt % to about 60 wt %, from about 10 wt % to about 50 wt %, from about 10 wt % to about 40 wt % from about 12 wt % to about 38 wt %, from about 15 wt % to about 35 wt %, from about 16 wt % to about 34 wt %, from about 30 wt % to about 40 wt %, from about 30 wt % to about 35 wt %, from about 33 wt % to about 35 wt %, about 32 wt %, about 33 wt %, about 34 wt %, about 35 wt %, from about 10 wt % to about 20 wt %, from about 13 wt % to about 18 wt %, from about 16 wt % to about 18 wt %, about 15 wt %, about 16 wt %, about 17 wt %, about 18 wt %, from about 40 wt % to about 50 wt %, from about 46 wt % to about 49 wt %, about 46 wt %, about 47 wt %, or about 48 wt %. In one alternative, the amount of HPMC-AS present in the solid dispersion is about 46 wt % to about 49 wt %, about 46 wt %, about 47 wt %, about 48 wt %, from about 33 wt % to about 35 wt %, about 33 wt %, about 34 wt %, about 35 wt %, from about 16 wt % to about 34 wt %, from about 16 wt % to about 18 wt %, about 16 wt %, about 17 wt %, or about 18 wt %. In another alternative, the amount of HPMC-AS present in the solid dispersion is about 46 wt %, about 47 wt %, about 48 wt %, about 33 wt %, about 34 wt %, about 35 wt %, about 16 wt %, about 17 wt %, or about 18 wt % HPMC-AS. In yet another alternative, the amount of HPMC-AS present in the solid dispersion is about 46 wt %, about 47 wt %, or about 48 wt %.

In a third embodiment, the solid dispersion comprises equal amounts of rifaximin and polymer. Thus, for example, the solid dispersion comprises from about 10 wt % to about 60 wt %, from about 10 wt % to about 50 wt %, from about 10 wt % to about 40 wt %, from about 12 wt % to about 38 wt %, from about 15 wt % to about 35 wt %, from about 16 wt % to about 34 wt %, from about 30 wt % to about 40 wt %, from about 30 wt % to about 35 wt %, from about 33 wt % to about 35 wt %, about 32 wt %, about 33 wt %, about 34 wt %, about 35 wt %, from about 10 wt % to about 20 wt %, from about 13 wt % to about 18 wt %, from about 16 wt % to about 18 wt %, about 15 wt %, about 16 wt %, about 17 wt %, about 18 wt %, from about 40 wt % to about 50 wt %, from about 46 wt % to about 49 wt %, about 46 wt %, about 47 wt %, or about 48 wt % rifaximin and HPMC-AS. In another aspect, the solid dispersion comprises from about 46 wt % to about 49 wt %, about 46 wt %, about 47 wt %, about 48 wt %, from about 33 wt % to about 35 wt %, about 33 wt %, about 34 wt %, about 35 wt %, from about 16 wt % to about 34 wt %, from about 16 wt % to about 18 wt %, about 16 wt %, about 17 wt %, or about 18 wt % rifaximin and HMPC-AS. In another aspect, the solid dispersion comprises about 46 wt %, about 47 wt %, about 48 wt %, about 33 wt %, about 34 wt %, about 35 wt %, about 16 wt %, about 17 wt %, or about 18 wt % rifaximin and HPMC-AS. In yet another aspect, the solid dispersion comprises about 46 wt %, about 47 wt %, or about 48 wt % rifaximin and HPMC-AS.

In a fourth embodiment, the solid dispersion comprising rifaximin and HPMC-AS further comprises poloxamer 407 (e.g., Pluronic® F-127), wherein the remaining components and amounts present in the solid dispersion are as described in the second or third embodiment.

In a fifth embodiment, the solid dispersion comprising rifaximin and HPMC-AS further comprises poloxamer 407 (e.g., Pluronic® F-127) in an amount from about 0.5 wt % to about 7 wt %, from about 0.5 wt % to about 5 wt %, from about 1 wt % to about 5 wt %, from about 1 wt % to about 4 wt %, from about 2 wt % to about 4 wt %, from about 4 wt % to about 6 wt %, from about 3 wt % to about 5 wt %, from about 2 wt % to about 4 wt %, from about 1 wt % to about 2 wt %, about 1 wt %, about 2 wt %, about 3 wt %, about 4 wt %, about 5 wt %, about 5.5 wt %, or about 6 wt %, wherein the remaining components and amounts present in the solid dispersion are as described in the second, third, or fourth embodiment. In one alternative, the solid dispersion comprises about 1 wt %, about 2 wt %, about 3 wt %, about 4 wt %, about 5 wt %, about 5.5 wt %, about 5.6 wt %, about 5.7 wt %, or about 6 wt % poloxamer 407 (e.g., Pluronic® F-127), wherein the remaining components and amounts present in the solid dispersion are as described in the second, third, or fourth embodiment. In yet another alternative, the solid dispersion of rifaximin comprises about 5 wt %, about 5.5 wt %, or about 6 wt % poloxamer 407 (e.g., Pluronic® F-127), wherein the remaining components and amounts present in the solid dispersion are as described in the second, third, or fourth embodiment.

In a sixth embodiment, provided are pharmaceutical compositions comprising the solid dispersion of any one of the first, second, third, fourth, or fifth embodiment.

In a seventh embodiment, provided are pharmaceutical compositions comprising the solid dispersions of any one of the first, second, third, fourth, or fifth embodiments together with croscarmellose sodium (crosslinked carboxymethyl cellulose sodium).

In an eighth embodiment, provided are pharmaceutical compositions comprising the solid dispersions of any one of the first, second, third, fourth, or fifth embodiments together with croscarmellose sodium in an amount from about 2 wt % to about 15 wt %, from about 3 wt % to about 14 wt %, from about 4 wt % to about 14 wt %, from about 2 wt % to about 13 wt %, from about 3 wt % to about 13 wt %, from about 4 wt % to about 13 wt %, from about 11 wt % to about 14 wt %, from about 12 wt % to about 14 wt %, from about 4 wt % to about 10 wt %, about 12 wt %, about 12.5 wt %, about 13 wt %, about 13.5 wt %, from about 4 wt % to about 6 wt %, about 5 wt %, from about 8% to about 10 wt %, or about 9 wt % based on the total amount (wt %) of components in the pharmaceutical composition, wherein the remaining components and amounts present in the pharmaceutical composition include and are as described in the second, third, fourth, fifth, sixth, or seventh embodiment. In one alternative, the croscarmellose sodium is present in an amount from about 4 wt % to about 14 wt %, from about 12 wt % to about 14 wt %, about 13 wt %, from about 4 wt % to about 6 wt %, about 5 wt %, from about 8% to about 10 wt %, or about 9 wt % based on the total amount (wt %) of components in the pharmaceutical composition, wherein the remaining components and amounts present in the pharmaceutical composition include and are as described in the second, third, fourth, fifth, sixth, or seventh embodiment. In another alternative, the croscarmellose sodium is present in an amount from of rifaximin is about 13 wt %, about 5 wt %, or about 9 wt % based on the total amount (wt %) of components in the pharmaceutical composition, wherein the remaining components and amounts present in the pharmaceutical composition include and are as described in the second, third, fourth, fifth, sixth, or seventh embodiment.

In a ninth embodiment, the pharmaceutical compositions described herein further comprise microcrystalline cellulose, wherein the remaining components and amounts present in the pharmaceutical composition include and are as described in the second, third, fourth, fifth, sixth, seventh, or eighth embodiment.

In a tenth embodiment, the pharmaceutical compositions described herein further comprise microcrystalline cellulose present in an amount from about 5 wt % to about 60 wt %, from about 10 wt % to about 55 wt %, from about 5 wt % to about 15 wt %, from about 8 wt % to about 13 wt %, from about 10 wt % to about 12 wt %, from about 10 wt % to about 19 wt %, about 11 wt %, from about 15 wt % to about 25 wt %, from about 17 wt % to about 19 wt %, about 18 wt %, from about 40 wt % to about 60 wt %, from about 45 wt % to about 55 wt %, from about 49 wt % to about 55 wt %, from about 49 wt % to about 51 wt %, from about 53 wt % to about 55 wt %, about 50 wt %, or about 54 wt % based on the total amount (wt %) of components in the pharmaceutical composition, wherein the remaining components and amounts present in the pharmaceutical composition include and are as described in the second, third, fourth, fifth, sixth, seventh, eighth, or ninth embodiment.

In an eleventh embodiment, the pharmaceutical compositions described herein further comprise colloidal silicon dioxide, wherein the remaining components and amounts present in the pharmaceutical composition include and are as described in the second, third, fourth, fifth, sixth, seventh, eighth, ninth, or tenth embodiment.

In a twelfth embodiment, the pharmaceutical compositions described herein further comprise colloidal silicon dioxide present in an amount from about 0.1 wt % to about 0.3 wt %, from about 0.15 wt % to about 0.25 wt %, or about 0.2 wt % based on the total amount (wt %) of components in the pharmaceutical composition, wherein the remaining components and amounts present in the pharmaceutical composition include and are as described in the second, third, fourth, fifth, sixth, seventh, eighth, ninth, tenth, or eleventh embodiment.

In a thirteenth embodiment, the pharmaceutical compositions described herein further comprise magnesium stearate, wherein the remaining components and amounts present in the pharmaceutical composition include and are as described in the second, third, fourth, fifth, sixth, seventh, eighth, ninth, tenth, eleventh, or twelfth embodiment.

In a fourteenth embodiment, the pharmaceutical compositions described herein further comprise magnesium stearate present in an amount from about 0.3 wt % to about 0.6 wt %, from about 0.4 wt % to about 0.6 wt %, from about 0.45 wt % to about 0.55 wt %, about 0.45 wt %, about 0.47 wt %, or about 0.49 wt % based on the total amount (wt %) of components in the pharmaceutical composition, wherein the remaining components and amounts present in the pharmaceutical composition include and are as described in the second, third, fourth, fifth, sixth, seventh, eighth, ninth, tenth, eleventh, twelfth, or thirteenth embodiment.

In a fifteenth embodiment, provided is a provided is a pharmaceutical composition comprising from about 33 wt % to about 35 wt % rifaximin; from about 33 wt % to about 35 wt % HPMC-AS; from about 3 wt % to about 5 wt % poloxamer 407; from about 4 wt % to about 14 wt % croscarmellose sodium; from about 10 wt % to about 19 wt % microcrystalline cellulose; from about 0.15 wt % to about 0.25 wt % colloidal silicon dioxide; and from about 0.45 wt % to about 0.55 wt % magnesium stearate.

In a sixteenth embodiment, provided is a pharmaceutical composition according to the fifteenth embodiment, wherein comprising the croscarmellose sodium is present in an amount of from about 12 wt % to about 14 wt %. Alternatively, provided is a pharmaceutical composition according to the fifteenth embodiment, wherein comprising the croscarmellose sodium is present in an amount of about 13%.

In a seventeenth embodiment, provided is a pharmaceutical composition according to the fifteenth or sixteenth embodiment wherein the microcrystalline cellulose is present in an amount from about 10 wt % to about 12 wt %. Alternatively, provided is a pharmaceutical composition according to the fifteenth or sixteenth embodiment wherein the microcrystalline cellulose is present in an amount of about 11 wt %.

In an eighteenth embodiment, provided is a pharmaceutical composition according to the fifteenth embodiment, wherein the croscarmellose sodium is present in an amount from about 4 wt % to about 6 wt %. Alternatively, provided is a pharmaceutical composition according to the fifteenth embodiment, wherein the croscarmellose sodium is present in an amount of about 5 wt %.

In a nineteenth embodiment, provided is a pharmaceutical composition according to the fifteenth or eighteenth embodiment, wherein the microcrystalline cellulose is present in an amount from about 17 wt % to about 19 wt %. Alternatively, provided is a pharmaceutical composition according to the fifteenth or eighteenth embodiment, wherein the microcrystalline cellulose is present in an amount of about 18 wt %.

In a twentieth embodiment, provided is a pharmaceutical composition according to the fifteenth, sixteenth, seventeenth, eighteenth, or nineteenth embodiment, wherein the poloxamer 407 is present in an amount of about 4%.

In a twenty-first embodiment, provided is a pharmaceutical composition according to the fifteenth, sixteenth, seventeenth, eighteenth, nineteenth, or twentieth embodiment, wherein the colloidal silicon dioxide is present in an amount of about 0.20 wt %.

In a twenty-second embodiment, provided is a pharmaceutical composition according to the fifteenth, sixteenth, seventeenth, eighteenth, nineteenth, twentieth, or twenty-first embodiment, wherein the magnesium stearate is present in an amount of about 0.50 wt %.

In a twenty-third embodiment, provided is a pharmaceutical composition according to the fifteenth, sixteenth, seventeenth, eighteenth, nineteenth, twentieth, twenty-first, or twenty-second embodiment, wherein the rifaximin is present in an amount of about 34%.

In a twenty-fourth embodiment, provided is a pharmaceutical composition according to the fifteenth, sixteenth, seventeenth, eighteenth, nineteenth, twentieth, twenty-first, twenty-second, or twenty-third embodiment, wherein the HPMC-AS is present in an amount of about 34%.

In a twenty-fifth embodiment, provided is a pharmaceutical composition according to the fifteenth, sixteenth, seventeenth, eighteenth, nineteenth, twentieth, twenty-first, twenty-second, twenty-third embodiment, or twenty fourth embodiment, wherein the total amount of rifaximin is about 80 mg.

In a twenty-sixth embodiment, provided is a pharmaceutical composition comprising from about 16 wt % to about 18 wt % rifaximin; from about 16 wt % to about 18 wt % HPMC-AS; from about 1 wt % to about 2 wt % poloxamer 407; from about 4 wt % to about 10 wt % croscarmellose sodium; from about 49 wt % to about 55 wt % microcrystalline cellulose; from about 0.15 wt % to about 0.25 wt % colloidal silicon dioxide; and from about 0.45 wt % to about 0.55 wt % magnesium stearate.

In a twenty-seventh embodiment, provided is a pharmaceutical composition according to the twenty-sixth embodiment, wherein the croscarmellose sodium is present in an amount from about 8 wt % to about 10 wt %. Alternatively, provided is a pharmaceutical composition according to the twenty-sixth embodiment, wherein the croscarmellose sodium is present in an amount of about 9 wt %.

In a twenty-eighth embodiment, provided is a pharmaceutical composition according to the twenty-sixth or twenty-seventh embodiment, wherein the microcrystalline cellulose is present in an amount from about 49 wt % to about 51 wt %. Alternatively, provided is a pharmaceutical composition according to the twenty-sixth or twenty-seventh embodiment, wherein the microcrystalline cellulose is present in an amount of about 51 wt %.

In a twenty-ninth embodiment, provided is a pharmaceutical composition according to the twenty-sixth embodiment, wherein the croscarmellose sodium is present in an amount from about 4 wt % to about 6 wt %. Alternatively, provided is a pharmaceutical composition according to the twenty-sixth embodiment, wherein the croscarmellose sodium is present in an amount of about 5 wt %.

In a thirtieth embodiment, provided is a pharmaceutical composition according to the twenty-sixth or twenty-ninth embodiment, wherein the microcrystalline cellulose is present in an amount from about 53 wt % to about 55 wt %. Alternatively, provided is a pharmaceutical composition according to the twenty-sixth or twenty-ninth embodiment, wherein the microcrystalline cellulose is present in an amount of about 54 wt %.

In a thirty-first embodiment, provided is a pharmaceutical composition according to the twenty-sixth, twenty-seventh, twenty-eighth, twenty-ninth, or thirtieth embodiment, wherein colloidal silicon dioxide is present in an amount of about 0.20 wt %.

In a thirty-second embodiment, provided is a pharmaceutical composition according to the twenty-sixth, twenty-seventh, twenty-eighth, twenty-ninth, thirtieth, or thirty-first embodiment, wherein the magnesium stearate is present in an amount of about 0.50 wt %.

In a thirty-third embodiment, provided is a pharmaceutical composition according to the twenty-sixth, twenty-seventh, twenty-eighth, twenty-ninth, thirtieth, thirty-first, or thirty-second embodiment, wherein the rifaximin is present in an amount of about 17 wt %.

In a thirty-fourth embodiment, provided is a pharmaceutical composition according to the twenty-sixth, twenty-seventh, twenty-eighth, twenty-ninth, thirtieth, thirty-first, thirty-second, or thirty-third embodiment, wherein the HMPC-AS is present in an amount of about 17 wt %.

In a thirty-fifth embodiment, provided is a pharmaceutical composition according to the twenty-sixth, twenty-seventh, twenty-eighth, twenty-ninth, thirtieth, thirty-first, thirty-second, thirty-third, or thirty-fourth embodiment, wherein the total amount of rifaximin is 40 mg.

In a thirty-sixth embodiment, the pharmaceutical compositions described herein are in the form of a tablet.

In a thirty-seventh embodiment, the pharmaceutical compositions described herein are in the form of a tablet, and are immediate release or sustained extended release. In one alternative, the pharmaceutical composition is a sustained extended release tablet.

In a thirty-eighth embodiment, the pharmaceutical compositions described herein are film coated. Coatings are known to those of skill in the art and may include immediate release or sustained release coatings. An example of a film coating is Opadry II Blue 85F90614 by Colorcon®.

Other solid dispersions and pharmaceutical compositions included in the present disclosure are described in the Exemplification section below.

### Uses, Formulation and Administration Dosage Forms

The invention described herein includes methods of treating liver diseases and disorders including methods of treating Overt Hepatic Encephalopathy (OHE) (e.g., preventing the occurrence of OHE) and methods of preventing complications of liver cirrhosis in patients, such as patients having early decompensation, by administering one or more solid dispersion pharmaceutical compositions described herein.

In some embodiments, the invention includes a method of using the disclosed solid dispersions pharmaceutical compositions to prevent complications of liver cirrhosis such as e.g., in subjects with early decompensation. Thus, provided are methods of using the disclosed solid dispersions pharmaceutical compositions to delay encephalopathy decompensation in cirrhosis.

In some embodiments, the invention described herein relates to delaying encephalopathy decompensation in subjects with cirrhosis comprising administering at least one (e.g., one) rifaximin SSD composition (e.g., tablet) described herein. According to other embodiments, the invention described herein relates to delaying encephalopathy decompensation in subjects with cirrhosis comprising administering at least one (e.g., one) 40 mg rifaximin SSD IR composition (e.g., tablet) described herein.

In some embodiments, the invention described herein relates to delaying encephalopathy decompensation in subjects with cirrhosis comprising administering at least one (e.g., one) rifaximin SSD composition (e.g., tablet) described herein, wherein the subject is characterized as having cirrhosis of the liver and medically controlled ascites.

In some embodiments, the invention described herein relates to delaying encephalopathy decompensation in subjects with cirrhosis comprising administering at least one (e.g., one) 40 mg rifaximin SSD IR composition (e.g., tablet) described herein, wherein the subject is characterized as having cirrhosis of the liver and medically controlled ascites.

In some embodiments, the invention described herein relates to delaying encephalopathy decompensation in subjects with cirrhosis comprising administering at least one (e.g., one) rifaximin SSD composition (e.g., tablet) described herein, wherein the subject is characterized as having cirrhosis of the liver and medically controlled ascites for a period of at least 30 days.

In some embodiments, the invention described herein relates to delaying encephalopathy decompensation in subjects with cirrhosis comprising administering at least one (e.g., one) 40 mg rifaximin SSD IR composition (e.g., tablet) described herein, wherein the subject is characterized as having cirrhosis of the liver and medically controlled ascites for a period of at least 30 days. In one aspect, the cirrhosis in the foregoing subject is determined by one or more of histopathological evidence of cirrhosis, transient elastography, the presence of esophageal varices, and thrombocytopenia characterized by a blood platelet level of less than 150,000 in a patient with chronic liver disease. In one aspect, the foregoing subjects have one or more of Child-Pugh B Classification score of from 7 to 9 without OHE prior to treatment, a Model End Stage Liver Disease-Sodium (MELD-Na) score of less than 15 prior to treatment, a Conn (West Haven Criteria) score of less than 2 prior to treatment, and a Mini-Mental State Examination (MMSE) score of at least 25 prior to treatment. In some embodiments, the foregoing subjects demonstrate no cognitive impairment prior to treatment. In one aspect, the foregoing subjects do not require paracentesis. In one aspect, the foregoing subject has a Conn score of 2 or higher, e.g., 2, 3, or 4.

Also provided herein are methods of using the solid dispersions and pharmaceutical compositions thereof to prevent all-cause mortality e.g., in subjects with liver cirrhosis who may also have early decompensation.

Also provided herein are methods of using the solid dispersions and pharmaceutical compositions thereof to reduce the time to hospitalization that is associated with complications of liver disease (e.g., liver cirrhosis complications) such as e.g., reducing the time to hospitalization from one or more of hepatic encephalopathy (HE), esophageal variceal bleeding (EVB), spontaneous bacterial peritonitis (SBP), and hepatorenal syndrome (HRS).

Also provided herein are methods of using the solid dispersions and pharmaceutical compositions thereof to prevent hospitalization that is associated with complications of liver disease (e.g., liver cirrhosis complications) such as e.g., reducing the time to hospitalization from one or more of hepatic encephalopathy (HE), esophageal variceal bleeding (EVB), spontaneous bacterial peritonitis (SBP), and hepatorenal syndrome (HRS).

Also provided herein are methods of using the solid dispersions and pharmaceutical compositions thereof to reduce the time to all-cause mortality that is associated with complications of liver disease (e.g., liver cirrhosis complications) such as e.g., reducing the time to all-cause mortality from one or more of hepatic encephalopathy (HE), esophageal variceal bleeding (EVB), spontaneous bacterial peritonitis (SBP), and hepatorenal syndrome (HRS).

Also provided herein are methods of using the solid dispersions and pharmaceutical compositions thereof to prevent all-cause mortality that is associated with complications of liver disease (e.g., liver cirrhosis complications) such as e.g., reducing the time to all-cause mortality from one or more of hepatic encephalopathy (HE), esophageal variceal bleeding (EVB), spontaneous bacterial peritonitis (SBP), and hepatorenal syndrome (HRS).

Further provided are methods of using the solid dispersions and pharmaceutical compositions thereof to reduce the time to development of refractory ascites in e.g., subjects having early decompensated liver cirrhosis or liver cirrhosis complications such as HE, EVB, SBP, or HRS.

Suitable dosage forms that can be used with the solid dispersions and compositions herein include, but are not limited to, capsules, tablets, mini-tablets, beads, beadlets, pellets, granules, granulates, and powder. Suitable dosage forms may be coated, for example using an enteric coating. In some embodiments, the solid dispersions and compositions are formulated as tablets, caplets, or capsules. In one embodiment, the solid dispersions and compositions are formulated as a tablet.

Provided compositions may be formulated such that a dosage of between 0.001-100 mg/kg body weight/day of the inhibitor can be administered to a patient receiving these compositions. It should also be understood that a specific dosage and treatment regimen for any particular patient will depend upon a variety of factors, including age, body weight, general health, sex, diet, time of administration, rate of excretion, drug combination, the judgment of the treating physician, and the severity of the particular disease being treated.

The amount of a provided dispersion in the composition will also depend upon the particular compound in the composition. In one aspect, the dosage amount of rifaximin in a provided composition is 40 mg. In another aspect, the dosage amount of rifaximin in a provided composition is 80 mg.

In an embodiment, the soluble solid dispersion tablet will include rifaximin in an amount of about 40 mg and will be an immediate release tablet. For example, in some embodiments, a patient may receive an 80 mg dosage of rifaximin through the administration of two tablets containing 40 mg of rifaximin.

In some embodiments, the rifaximin compositions described herein may be combined with lactulose, and the rifaximin composition in addition to lactulose may be administered to a patient in need thereof in accordance with the foregoing methods. Lactulose is a non-absorbable disaccharide that, without being limited to any one theory of the invention, may inhibit intestinal ammonia production and the conversion of lactulose to lactic acid and acetic acid results in the acidification of the gut lumen. In an embodiment, lactulose (with a rifaximin composition described herein) may be administered to a patient at a dose of about 25 mL (16.7 g) of oral syrup every 1 to 2 hours. In an embodiment, lactulose (with a rifaximin composition described herein) may be administered to a patient at a dose that provides about 2 to 3 soft bowel movements per day, which may be about 10 g to 30 g (15 mL to 45 mL) 2 to 4 times per day. In an embodiment, lactulose (with a rifaximin composition described herein) may be administered to a patient at an oral dosage of about 2 to 3 tablespoons (30 mL to 45 mL, containing 20 g to 30 g of lactulose) three times daily, and adjusting the dosage as necessary to produce 2 to 3 soft stools per day. In some embodiments, lactulose (with a rifaximin composition described herein) may be administered according to the foregoing dosages indefinitely in patients having recurrent or persistent hepatic encephalopathy. In some embodiments, were a patient is a comatose state, lactulose (with a rifaximin composition described herein) may be administered to the patient at a dose of about 300 mL in 1 L of water over 6 to 8 hours (either nasogastrically or rectally) until the patient is awake enough to start oral therapy.

### EXAMPLES

### Example 1. Preparation of Solid Dispersions and Methods of Using the Same General Preparation of Solid Dispersions

The solid dispersions described herein can be prepared by a number of methods, including by melting and solvent evaporation. The solid dispersions described herein can also be prepared according to the procedures described in: Chiou W L, Riegelman S: “Pharmaceutical applications of solid dispersion systems”, J. Pharm. Sci. 1971; 60: 1281-1302; Serajuddin A T M: “Solid dispersion of poorly water-soluble drugs: early promises, subsequent problems, and recent breakthroughs”, J. Pharm. Sci. 1999; 88: 1058-1066; Leuner C, Dressman J: “Improving drug solubility for oral delivery using solid dispersions”, Eur. J. Pharm. Biopharm. 2000; 50:47-60; and Vasconcelos T, Sarmento B, Costa P: “Solid dispersions as strategy to improve oral bioavailability of poor water soluble drugs”, Drug Discovery Today 2007; 12:1068-1075, all of which are incorporated herein by reference in their entireties.

In one aspect, the components, e.g., rifaximin, polymer and methanol are mixed and then spray dried. Exemplary conditions are summarized in Table 1 below.

Exemplary Spray Drying Process Parameters, include for example:
Spray Dryer—e.g., PSD 1;
Single or multi-fluid nozzle: e.g., a two Fluid Niro Nozzle;
Nozzle orifice—0.1-10 mm;
Inlet gas temperature—75-150±5 deg C.;
Process gas flow (mmH2O)—20-70, preferred 44;
Atomizing gas pressure—0.7-1 bar;
Feed rate—2-7 kg/Hr;
Outlet temperature—30-70±3 deg C.;
Solution temperature—20-50 deg C.; and
Post spray drying vacuum dry at 20-60 deg C., for between about 2 and 72 hrs.

TABLE 1

| | | | | Inlet temp. | Outlet temp. | Spray |
|---|---|---|---|---|---|---|
| Description | Inlet temp. | Aspirator | Pump | (measured, | (measured, | rate (b) |
| (a) | (set, ° C.) | % | % | ° C.) | ° C.) | mL/min |
| (50:50) | 120 | 95 | 40-30 | 120-119 | 60-45 | 9.6 |
| HPMC- | | | | | | |
| AS:rifaximin, | | | | | | |
| ~10 g scale | | | | | | |

(a) approximate ratio of rifaximin to HPMC-AS, by weight.
(b) flow rates are estimated at 30% pump.

A representative batch formula is provided in Table 2.

TABLE 2

| | | | Theoretical |
|---|---|---|---|
| | | | Quantity |
| Ingredient | Function | % w/w | (kg/batch) |
| Rifaximin | Active | 47.20 | 22.18 |
| HPMC-AS | Polymer | 47.20 | 22.18 |
| Poloxamer 407 | Surfactant | 5.60 | 2.64 |
| Methanol^{a} | Solvent | — | (438.0) |
| Nitrogen^{b} | Process gas | — | — |

| | | |
|---|---|---|
| Total Theoretical Weight | 100.00 | 47.00 |

^{a}Removed during drying process; ^{p}rocess gas for drying and atomization; not incorporated in product

### Blending/Encapsulation Procedure

The components and composition of an 80 mg and 40 mg immediate and sustained extended release tables are proved in Table 3 below.

TABLE 3

| | | Theoretical Quantity (mg/tablet) | | | |
|---|---|---|---|---|---|
| | | 80 mg- | 80 mg- | 40 mg- | 80 mg- |
| Ingredient | Function | IR | SER | IR | IR |
| Rifaximin | Active | 80 | 80 | 40 | 40 |
| HPMC-AS | Polymer | 80 | 80 | 40 | 40 |
| Poloxamer 407 | Surfactant | 9.49 | 9.49 | 4.75 | 4.75 |
| Croscarmellose | Dissolution | 30.15 | 11.33 | 20.74 | 11.33 |
| sodium | enhancer | | | | |
| Microcrystalline | Filler | 25.28 | 44.10 | 119.43 | 128.84 |
| cellulose | | | | | |
| Colloidal silicon | Glidant | 0.45 | 0.45 | 0.45 | 0.45 |
| dioxide | | | | | |
| Magnesium stearate | Lubricant | 1.13 | 1.13 | 1.13 | 1.13 |
| (non-bovine) | | | | | |
| Opadry II Blue | Coating | 11.92 | 11.92 | 11.92 | 11.92 |
| 85F90614 | | | | | |
| (PVA coating) | | | | | |
| Purified Water | Solvent for | — | — | — | — |
| | coating | | | | |
| | solution | | | | |

| | | | | |
|---|---|---|---|---|
| Total Theoretical Weight | 238.42 | 238.42 | 238.42 | 238.42 |

### Clinical Data

The following data was obtained using the compositions described in Table 3.

A Phase 2, randomized, double-blind, placebo-controlled, parallel multicenter study evaluation of the efficacy (prevention of hospitalization for complications of liver cirrhosis or all-cause mortality in subjects with early decompensation) and safety of rifaximin SSD tablets in subjects with early decompensated liver cirrhosis was conducted. Subjects with documented ascites who had not previously experienced SBP, EVB, or HRS were enrolled in the study. Subjects completed a 1 to 21-day Screening Period, a 24-week Treatment Period, and a 2-week Follow-up Period. Approximately 420 subjects who successfully completed the Screening Period were randomized in a 1:1:1:1:1:1 allocation to 1 of 6 treatment groups and entered the Treatment Period. All treatments were administered once daily at bedtime. Assessments of efficacy and safety were performed during clinic visits at Day 1 (baseline), Weeks 2, 4, 8, 12, 16, 20, and 24 (End of Treatment [EOT]). All subjects completed an End of Study (EOS) visit at Week 26 (or early termination if applicable) for final safety assessments.

### Inclusion Criteria

A subject was eligible for inclusion in this study if he/she met all of the following criteria:

1. Subject was ≥18 years of age.
2. Subject was male or female.
Females of childbearing (reproductive) potential had to have a negative serum pregnancy test at Screening and had to agree to use an acceptable method of contraception throughout their participation in the study. Acceptable methods of contraception included double barrier methods (condom with spermicidal jelly or a diaphragm with spermicide), hormonal methods (eg, oral contraceptives, patches or medroxyprogesterone acetate), or an intrauterine device (IUD) with a documented failure rate of less than 1% per year. Abstinence or partner(s) with a vasectomy could be considered an acceptable method of contraception at the discretion of the investigator. Note: Females who had been surgically sterilized (eg, hysterectomy or bilateral tubal ligation) or who were postmenopausal (total cessation of menses for >1 year) were not considered “females of childbearing potential.”
   3. Subject had a diagnosis of liver cirrhosis and documented ascites, either by imaging study or physical exam (Note: Subjects with Grade 1 ascites were permitted in the study), but had not yet experienced any of the following complications of cirrhosis:
EVB—clinically significant gastrointestinal bleed
SBP—greater than 250 polymorphonuclear (PMN) cells/mm³ and/or positive monomicrobial culture in the ascitic fluid
Renal failure in the presence of ascites—rise in the serum creatinine by 0.5 mg/dL (to greater than 1.5 mg/dL), with ascites documented on physical examination, imaging, and/or admitted on diuretics for the treatment of ascites
Development of medically refractory ascites.
   4. Subject had a MELD score of ≥12, a MELDNa score of ≥12, or a Child-Pugh B Classification (score=7-9).
   5. Subject was capable of understanding the requirements of the study, and was willing to comply with all study procedures.
   6. Subject understood the language of the informed consent form, and was capable and willing to sign the informed consent form.
   7. If applicable, subject had a close family member or other personal contact that could provide continuing oversight to the subject and was available to the subject during the conduct of the trial.

### Exclusion Criteria

A subject was not eligible for inclusion in this study if any of the following criteria applied:

1. Subject had a history of a major psychiatric disorder, including uncontrolled major depression or controlled or uncontrolled psychoses within the past 24 months prior to signing the informed consent (Diagnostic and Statistical Manual of Mental Disorders, 4th.) that, in the opinion of the investigator, would prevent completion of the study, interfere with analysis of study results, or negatively impact the subject's participation in the study.
2. Subject had history of alcohol abuse or substance abuse within the past 3 months prior to signing the informed consent (Diagnostic and Statistical Manual of Mental Disorders, 4th.).
3. Subject had documented primary sclerosing cholangitis (Note: subjects with primary biliary cirrhosis were allowed in the study).
4. Subject had undergone prophylactic variceal banding within 2 weeks of Screening or was scheduled to undergo prophylactic variceal banding during the study (Note: subjects with previous prophylactic variceal banding were allowed to participate in the study).
5. Subject had been diagnosed with an infection for which they are currently taking oral or parenteral antibiotics.
6. Subject had significant hypovolemia, or any electrolyte abnormality that could affect mental function (eg, serum sodium <125 mEq/L, serum calcium >10 mg/dL).
7. Subject had severe hypokalemia as defined by a serum potassium concentration <2.5 mEq/L.
8. Subject was anemic, as defined by a hemoglobin concentration of ≤8 g/dL.
9. Subject had renal insufficiency with a creatinine of ≥1.5 mg/dL.

Note: Laboratory tests related to Inclusion/Exclusion criteria could be repeated once, before considering subject as a Screening Failure (given all other Inclusion/Exclusion criteria are met/not met respectively) at the discretion of the Investigator.

10. Subject showed presence of intestinal obstruction or has inflammatory bowel disease.
11. Subject had Type 1 or Type 2 diabetes that was poorly controlled in the opinion of the investigator or had had an HbA1c>12% within the past 3 months prior to Screening or at the Screening visit.
12. Subject had a history of seizure disorders.
13. Subject had unstable cardiovascular or pulmonary disease, categorized by a worsening in the disease condition that required a change in treatment or medical care within 30 days of randomization.
14. Subject had an active malignancy within the last 5 years (exceptions: basal cell carcinomas of the skin, or if female, in situ cervical carcinoma that had been surgically excised).
15. Subject had hepatocellular carcinoma (HCC). Note: Alpha-fetoprotein (AFP) concentration was measured at Screening. If the AFP was greater than 200 ng/mL, the subject was excluded from participation in the study. If the AFP was above the upper limit of normal and ≤200 ng/mL, cross-sectional imaging or ultrasonography techniques had to be used to rule out HCC.
16. Subject had any condition or circumstance that adversely affected the subject or could cause noncompliance with treatment or visits, could affect the interpretation of clinical data, or could otherwise contraindicate the subject's participation in the study.
17. If female, subject was pregnant or at risk of pregnancy, or was lactating.
18. Known varicella, herpes zoster, or other severe viral infection within 6 weeks of randomization.
19. Known human immunodeficiency virus (HIV) infection.
20. Subject had a positive stool test for Yersinia enterocolitica, Campylobacter jejuni, Salmonella, Shigella, ovum and parasites, and/or Clostridium difficile.
NOTE: Results of stool tests had to be confirmed as negative prior to randomization.
21. Subject had a history of tuberculosis infection and/or had received treatment for a tuberculosis infection. If subject had a previous positive skin test for tuberculosis antigen then they were to have a current negative chest X-ray to be eligible and could not have received previous treatment.
22. Subject was an employee of the site that was directly involved in the management, administration, or support of this study or was an immediate family member of the same.
23. Subject had a history of hypersensitivity to rifaximin, rifampin, rifamycin antimicrobial agents, or any of the components of rifaximin SSD.
24. Subject used any investigational product or device, or participated in another research study within 30 days prior to randomization.
25. Subject had a documented overt HE episode (Conn score ≥2) that had not resolved within 30 days of Visit 1 (Screening).

### Treatments Administered

There were 6 treatment groups as listed below. The compositional components are presented above in Table 3. All treatments were to be administered orally qhs (at every bedtime). The duration of the treatment was 24 weeks.

Treatment A: rifaximin SSD 40 mg qhs (IR (immediate release) tablet)

Treatment B: rifaximin SSD 80 mg qhs (IR tablet)

Treatment C: rifaximin SSD 40 mg qhs (SER (sustained extended release) tablet)

Treatment D: rifaximin SSD 80 mg qhs (SER tablet)

Treatment E: rifaximin SSD 80 mg qhs (IR tablet)+rifaximin 80 mg qhs (SER tablet)

Treatment F: Placebo qhs

### Primary Efficacy Endpoints

Over the 24-week treatment period, the primary efficacy endpoint for the study was time to:
All-cause mortality, or
Hospitalization that was associated with 1 of the following complications of liver disease:
   HE—altered mental status diagnosed as HE, and defined as an increase of the Conn score to Grade ≥2 (ie, 0 or 1 to ≥2).
   EVB—occurrence of a clinically significant gastrointestinal bleed was defined as:
      Bleeding from an esophageal or gastric varix at the time of endoscopy, or
      The presence of large varices with blood evident in the stomach, and no other identifiable cause of bleeding observed during endoscopy.
      In addition, 1 or more of the following criteria had to be present:
         Drop in hemoglobin of greater than 2 g/dL over the first 48 hours post hospital admission,
         Transfusion requirement of 2 units of blood or more within 24 hours of hospital admission,
         A systolic blood pressure of less than 100 mm Hg, or
         Pulse rate greater than 100 beat/min at the time of admission.
            Note: Baveno IV criteria was also used to further define variceal bleeding episodes.
SBP—greater than 250 PMNcells/mm³ and/or positive monomicrobial culture in the ascitic fluid.
HRS was defined as:
   Progressive rise in serum creatinine (>1.5 mg/dL) with no improvement after at least 2 days with diuretic withdrawal and volume expansion with albumin;
   Absence of parenchymal kidney disease;
   Oliguria;
   Absence of shock; and
   No current or recent (within 3 months prior randomization) treatment with nephrotoxic drugs.

### Key Secondary Efficacy Endpoints

The key secondary efficacy endpoints of this study were overall hospitalization rate for each of the individual component of the primary endpoint or all-cause mortality over the 24-week treatment period.

### Other Secondary Endpoints

Other secondary endpoints of this study were the following:
Time to first hospitalization or all-cause mortality for each individual component of the primary endpoint.
All-cause hospitalization rate over the 24-week Treatment Period.
Liver cirrhosis mortality over the 24-week Treatment Period.
Time to development of medically refractory ascites, defined as ascites which could either no longer be effectively managed by:
   A low sodium diet and maximal doses of diuretics (up to 400 mg spironolactone and 160 mg furosemide per day), or
   Diuretics, due to the inability to tolerate side effects of maximal doses of diuretics.
Hospitalizations over the 24-week treatment period for all other infections.
Hospitalization as the result of Acute Kidney Injury (AKI) that was not attributable to HRS and was defined by a rapid reduction (over less than 48 hours) of kidney function as evidenced by:
   A rise in serum creatinine, (with either an absolute increase in serum creatinine of ≥0.3 mg/dL (≥26.4 μmol/L) or percentage increase in serum creatinine of ≥50%), and
   A reduction in urine output (defined as <0.5 ml/kg/hr for more than 6 hours).
Change in indices of Health Outcomes (Chronic Liver Disease Questionnaire (CLDQ_, Gastrointestinal Symptom Rating Scale (GSRS), Caregiver Burden Inventory (CBI), Epworth Sleepiness Scale (ESS)) at Weeks 4, 8, 12, 16, 20 and 24.
Pharmacokinetics of rifaximin and 25-desacetyl rifaximin assessing effects on factors including hepatic impairment, renal impairment and concomitant medications.
The critical flicker frequency (CFF) was assessed for each subject. CFF was assessed using a specialized CFF instrument.
Changes from baseline in blood ammonia concentrations at Weeks 2, 4, 8, 12, 16, 20 and 24.
Change from baseline in MELD (Model for End-Stage Liver Disease) and MELDNa (model end stage liver disease sodium) score at Weeks 2, 4, 8, 12, 16, 20 and 24.
Change from baseline in Child-Pugh score at Weeks 2, 4, 8, 12, 16, 20 and 24.

### Drug Concentration Measurements

Rifaximin and metabolite concentration data was collected according to the Full Population PK Sampling design recommended in the FDA Guidance for Industry: Population Pharmacokinetics.

### Disposition of Subjects

A total of 518 subjects were randomized in the study, of which 408 (78.8%) completed the study:
64 subjects in the 40 mg qhs IR group,
63 subjects in the 80 mg qhs IR group,
68 subjects in the 40 mg qhs SER group,
68 subjects in the 40 mg qhs SER group, 7
2 subjects in the 80 mg qhs SER group,
66 subjects in the combined IR/SER group and
75 subjects in the placebo group.

In total, 109 (21.0%) subjects prematurely discontinued from the study, with the largest number of discontinuations observed in the 80 mg qhs IR group (30.4%). The most common reason of premature discontinuation reported in the study was “withdrawal by subject”; this accounted for the premature discontinuation of 44 (8.5%) of all subjects that were randomized. This was followed by “death” which accounted for the premature discontinuation of 21 (4.1%) of all randomized subjects. Of all treatment groups, the 80 mg qhs IR group experienced the most number of premature discontinuations from the study (28 subjects in total), with “withdrawal by subject” reported as the most common reason of premature discontinuation (n=9).

### Data Sets Analyzed

Two datasets were analyzed: ITT population and PP populations.
ITT population was defined as all randomized subjects who took at least 1 dose of study drug.
PP population was defined as all subjects in the ITT population with the exception of those who failed to meet inclusion criteria 3 or 4, or meet exclusion criterion 1.
Safety population included all randomized subjects who took at least 1 dose of study drug.

The analyses of baseline characteristics and efficacy were performed on the ITT population. The primary efficacy analyses were also performed on the PP population as a sensitivity analysis.

### Analysis of Efficacy

The primary efficacy endpoint was the time to all-cause mortality or hospitalization that was associated with 1 of the following complications of liver disease: HE, EVB, SBP, or HRS over the 24-week treatment period was performed on the ITT population.

The primary analysis of time to hospitalization for any of the liver cirrhosis complications or all-cause mortality specified for the primary endpoint utilized a log-rank test stratified by analysis region (2-sided test at a significance level of 0.05).

Pairwise treatment group comparisons (each of the rifaximin SSD groups versus placebo) utilizing the log-rank test was also performed.

Additionally, Kaplan-Meier methods were used to estimate the proportion of subjects experiencing hospitalization for any of the liver cirrhosis complications or all-cause mortality on Days 28, 56, 84, 112, 140, and 168 for each treatment group.

Other analyses of the primary efficacy endpoint include sensitivity analyses (primary efficacy endpoint analyses using PP population) and prespecified subgroup analyses.

### Time to Hospitalization for any of the Liver Cirrhosis Complications or all-Cause Mortality

The primary efficacy endpoint was the time to all-cause mortality or hospitalization that was associated with 1 of the following complications of liver disease: HE, EVB, SBP, or HRS over the 24-week treatment period. Subjects who terminated early for reasons other than death were contacted approximately 24 weeks from randomization to determine if they experienced the primary endpoint. In the case of a subject's death, the subject's caregiver (if applicable) was contacted.

The time to hospitalization for any of the liver cirrhosis complications or all-cause mortality was defined as the duration between the date of first dose of the study drug and the date of first hospitalization for any of the liver cirrhosis complications or all-cause mortality.

Subjects who completed the entire 24-week treatment period without death or meeting the definition of liver cirrhosis complications of HE, EVB, SBP, or HRS were censored at the date of final visit (date of last contact). Subjects who prematurely discontinued before the end of the 24-week treatment period for reasons other than death were contacted monthly via a follow-up phone call for capture of cirrhosis complications, hospitalization, or death information. Subjects who did not meet the primary endpoint were censored at the date of last contact.

### Primary Efficacy Analysis

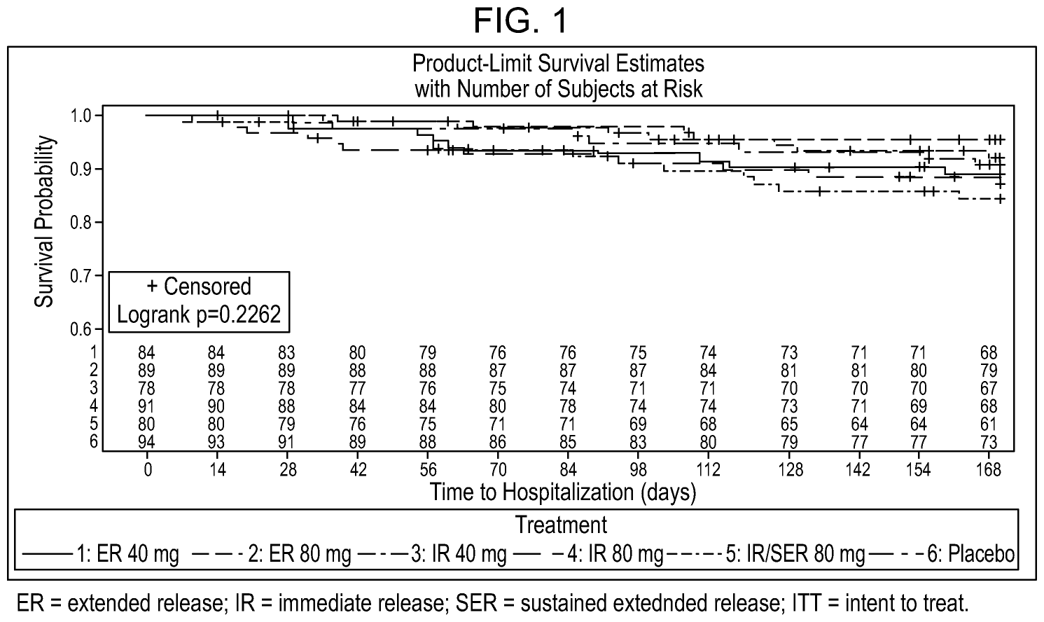

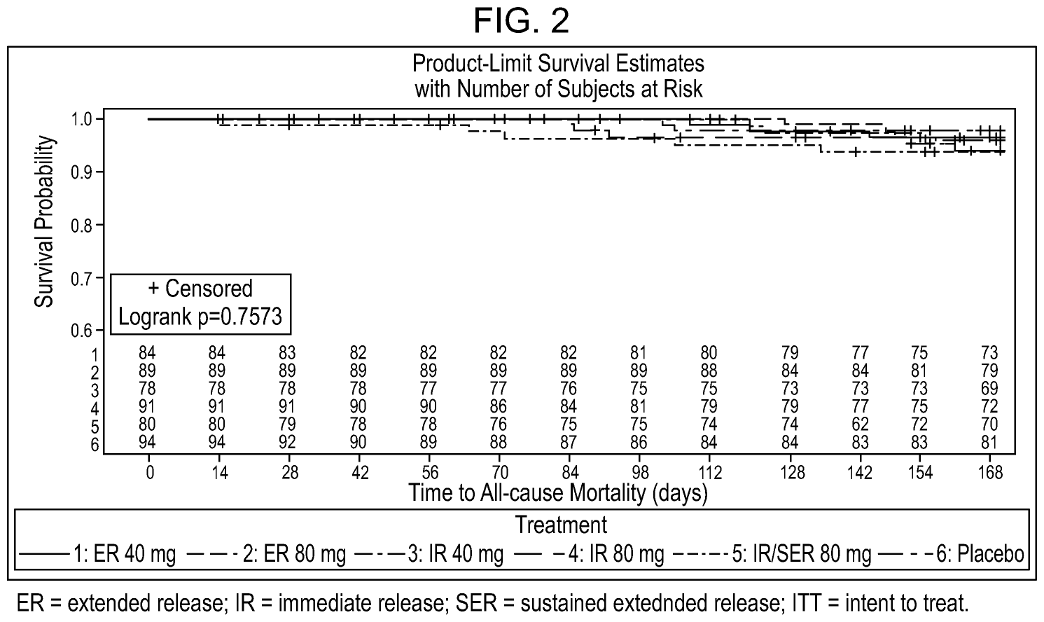

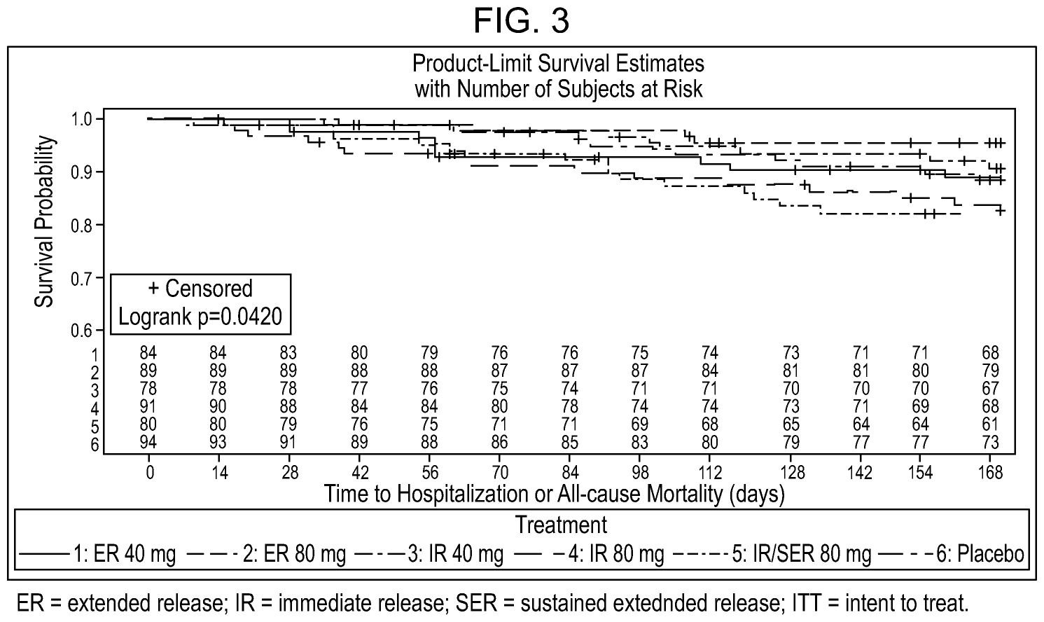

The primary analysis did not demonstrate an overall statistically significant difference in time to hospitalization for any of the liver cirrhosis complications or all-cause mortality up to 24 weeks in any group. The overall treatment comparison effect for any of the rifaximin SSD treatments versus placebo was not statistically significant (stratified log-rank p=0.8062) (Table 4). FIG. 1 presents a Kaplan-Meier estimate for the distribution of time to hospitalization for any of the liver cirrhosis complications by treatment group for the ITT population. Based on the Kaplan-Meier estimates, the SER 80 mg qhs treatment group presented with the highest survival rate of all treatment groups and the combined IR/SER treatment group had the lowest survival rate; however this effect was not statistically significant (log-rank p=0.2262). FIG. 2 presents a Kaplan-Meier estimate for the distribution of time to all-cause mortality by treatment group for the ITT population. Based on the Kaplan-Meier estimates, the placebo group presented with the highest survival rate followed by the SER 80 mg qhs treatment group and the combined IR/SER treatment group had the lowest survival rate; however this effect was not statistically significant (log-rank p=0.7573). FIG. 3 presents a Kaplan-Meier estimate for the distribution of time to hospitalization for any of the liver cirrhosis complications or all-cause mortality by treatment group for the ITT population. Based on the Kaplan-Meier estimates, the SER 80 mg qhs treatment group presented with the highest survival rate and the combined IR/SER treatment group had the lowest survival rate; this effect was statistically significant (log-rank p=0.0420).

### Supportive Analysis Based on the PP Population

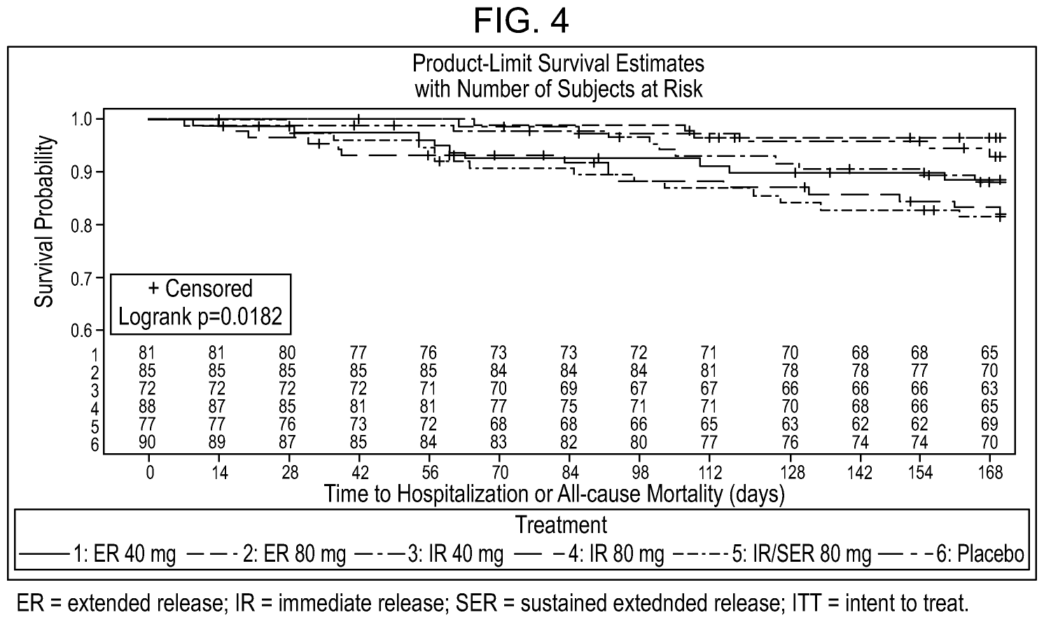

The results on the primary efficacy analysis based on the PP population were not consistent with the pairwise comparisons based on the ITT population (Table 4). The primary analysis on the PP population demonstrated a statistically significant difference in the time to hospitalization for any of the liver cirrhosis complications or all-cause mortality up to 24 weeks that was in favor of the SER 80 mg qhs treatment group versus placebo (stratified log-rank p=0.0464). There were no other statistically significant pairwise comparisons observed between the remaining active treatment groups and placebo (Table 5). The overall treatment comparison effect for any of the rifaximin SSD treatments versus placebo was not statistically significant (stratified log-rank p=0.9879). FIG. 4 presents a Kaplan-Meier estimate for the distribution of time to hospitalization for any of the liver cirrhosis complications or all-cause mortality by treatment group for the PP population. Based on the Kaplan-Meier estimates, the SER 80 mg qhs treatment group presented with the highest survival rate and the combined IR/SER treatment group had the lowest survival rate; this effect was statistically significant (log-rank p=0.0182).

TABLE 4
Time to Hospitalization for any of the Liver Cirrhosis Complications
or All-cause Mortality up to 24 Weeks - ITT Population

| | | | Censored | | |
|---|---|---|---|---|---|
| | # of Subjects | # of Events | <Week 24 | Week 24¹ | p-value² |
| Overall Treatment Comparison³ | | | | | |
| Any Rifaximin SSD Treatment | 422 | 50 (11.8%) | 31 (7.3%) | 341 (80.8%) | 0.8062 |
| Placebo | 94 | 10 (10.6%) | 11 (11.7%) | 73 (77.7%) | |
| Pairwise Comparisons | | | | | |
| (versus Placebo)³ | | | | | |
| Treatment A: Rifaximin SSD | 78 | 7 (9.0%) | 5 (6.4%) | 66 (84.6%) | 0.6316 |
| 40 mg qhs (IR Tablet) | | | | | |
| Treatment B: Rifaximin SSD | 91 | 15 (16.5%) | 9 (9.9%) | 67 (73.6%) | 0.2283 |
| 80 mg qhs (IR Tablet) | | | | | |
| Treatment C: Rifaximin SSD | 84 | 9 (10.7%) | 7 (8.3%) | 68 (81.0%) | 0.9666 |
| 40 mg qhs (SER Tablet) | | | | | |
| Treatment D: Rifaximin SSD | 89 | 4 (4.5%) | 6 (6.7%) | 79 (88.8%) | 0.0991 |
| 80 mg qhs (SER Tablet) | | | | | |
| Treatment E: Rifaximin SSD | 80 | 15 (18.8%) | 4 (5.0%) | 61 (76.3%) | 0.1792 |
| 80 mg qhs (IR Tablet) and | | | | | |
| Rifaximin SSD 80 mg qhs | | | | | |
| (SER Tablet) | | | | | |

IR = immediate release; ITT = intent to treat; qhs = once daily at bedtime; SER = sustained extended release; SSD = solid soluble dispersion.
¹Number of subjects censored at Week 24 (subject did not experience an event and was enrolled in the study at Week 24).
²P-value was obtained using a stratified log-rank test.
³Stratified by analysis region (study centers are grouped within 2 regions, centers in the United States and centers in Russia)

TABLE 5
Time to Hospitalization for any of the Liver Cirrhosis Complications
or All-cause Mortality up to 24 Weeks - PP Population

| | | | Censored | | |
|---|---|---|---|---|---|
| | # of Subjects | # of Events | <Week 24 | Week 24² | p-value³ |
| Overall Treatment Comparison⁴ | | | | | |
| Any Rifaximin SSD Treatment | 403 | 46 (11.4%) | 31 (7.7%) | 326 (80.9%) | 0.9879 |
| Placebo | 90 | 10 (11.1%) | 10 (11.1%) | 70 (77.8%) | |
| Pairwise Comparisons | | | | | |
| (versus Placebo)⁴ | | | | | |
| Treatment A: Rifaximin SSD | 72 | 5 (6.9%) | 5 (6.9%) | 62 (86.1%) | 0.3116 |
| 40 mg qhs (IR Tablet) | | | | | |
| Treatment B: Rifaximin SSD | 88 | 15 (17.0%) | 9 (10.2%) | 64 (72.7%) | 0.2247 |
| 80 mg qhs (IR Tablet) | | | | | |
| Treatment C: Rifaximin SSD | 81 | 9 (11.1%) | 7 (8.6%) | 65 (80.2%) | 0.9641 |
| 40 mg qhs (SER Tablet) | | | | | |
| Treatment D: Rifaximin SSD | 85 | 3 (3.5%) | 6 (7.1%) | 76 (89.4%) | 0.0464 |
| 80 mg qhs (SER Tablet) | | | | | |
| Treatment E: Rifaximin SSD | 77 | 14 (18.2%) | 4 (5.2%) | 59 (76.6%) | 0.2523 |
| 80 mg qhs (IR Tablet) and | | | | | |
| Rifaximin SSD 80 mg qhs | | | | | |
| (SER Tablet) | | | | | |

IR = immediate release; ITT = intent to treat; PP = per protocol; qhs = once daily at bedtime; SER = sustained extended release; SSD = solid soluble dispersion.
¹All subjects in the ITT population except those that failed inclusion criteria 3, 4 or met exclusion criterion 1.
²Number of subjects censored at Week 24 (subject did not experience an event and was enrolled in the study at Week 24.
³P-value was obtained using a stratified log-rank test.
⁴Stratified by analysis region (study centers are grouped within 2 regions, centers in the United States and centers in Russia).

### Prespecified Subgroup Analyses of the Primary Efficacy Endpoint

### Baseline MELD Category

The influence of a subject's baseline MELD category on the primary efficacy analysis was evaluated. Baseline MELD subgroups were categorized as MELD scores of ≤10, 11 to 18, 19 to 24, or ≥25. None of the pairwise comparisons versus placebo were statistically significant in any of the subgroups. The overall treatment comparison effect for any of the rifaximin SSD treatment versus placebo was not statistically significant (MELD score of ≤10: stratified log-rank p=0.8486; MELD score: 11 to 18 stratified log-rank p=0.7937; MELD score of 19 to 24: stratified log-rank p=0.3154; and MELD score of ≥25: stratified log-rank p=not applicable [1 event out of 1 subject]).

### Baseline MELDNa Category

The influence of a subject's baseline MELDNa category on the primary efficacy analysis was evaluated. Baseline MELDNa subgroups were categorized as MELDNa scores of ≤10, 11 to 18, 19 to 24, or ≥25. None of the pairwise comparisons versus placebo were statistically significant in any of the subgroups. The overall treatment comparison effect for any of the rifaximin SSD treatment versus placebo was not statistically significant (MELDNa score of ≤10: stratified log-rank p=0.3200; MELDNa score: 11 to 18 stratified log-rank p=0.9368; MELDNa score of 19 to 24: stratified log-rank p=0.2608; and MELDNa score of ≥25: stratified log-rank p=not applicable (3 events out of 4 subjects)).

### Baseline Child-Pugh Classification

The influence of a subject's baseline Child-Pugh classification on the primary efficacy analysis was evaluated. The baseline Child-Pugh classification subgroups were categorized as Class A, Class B, or Class C. None of the pairwise treatment comparisons versus placebo were statistically significant in any of the subgroups. The overall treatment comparison effect for any of the rifaximin SSD treatments versus placebo was not statistically significant (Class A: stratified log-rank p=not applicable (zero events); Class B: stratified log-rank p=0.7942 and Class C: stratified log-rank p=0.9516).

### Baseline Conn Score

The influence of a subject's baseline Conn score on the primary efficacy analysis was evaluated. Baseline Conn score subgroups were categorized as 0, 1, or 2. Table 6 presents the analysis of the primary efficacy endpoint by baseline Conn score. Consistent with the results of the PP population, a statistically significant difference in the time to hospitalization for any of the liver cirrhosis complications or all-cause mortality was observed within the Conn score subgroup 0 and was in favor of the SER 80 mg qhs treatment group versus placebo (stratified log-rank p=0.0460). This statistical significance was not evident within the Conn score subgroups 1 or 2 (although, subgroup 2 had 1 event out of 2 subjects).

The overall treatment comparison effect for any of the rifaximin SSD treatments versus placebo was not statistically significant for any subgroup (Conn score 0: stratified log-rank p=0.8915; Conn score 1: stratified log-rank p=0.8251; Conn score 2: not applicable [1 event out of 2 subjects]).

TABLE 6
Analysis of Primary Efficacy Endpoint: Time to Hospitalization for
any of the Liver Cirrhosis Complications or All-cause Mortality
by Baseline Conn Score up to 24 Weeks (Day 170) - ITT Population

| | | | Censored | | |
|---|---|---|---|---|---|
| | # of Subjects | # of Events | <Week 24 | Week 24¹ | p-value² |
| Conn Score: 0 | | | | | |
| Overall Treatment Comparison³ | | | | | |
| Any Rifaximin SSD Treatment | 260 | 34 (13.1%) | 20 (7.7%) | 206 (79.2%) | 0.8915 |
| Placebo | 57 | 7 (12.3%) | 6 (10.5%) | 44 (77.2%) | |
| Pairwise Comparisons | | | | | |
| (versus Placebo)³ | | | | | |
| Treatment A: Rifaximin SSD | 48 | 7 (14.6%) | 3 (6.3%) | 38 (79.2%) | 0.7477 |
| 40 mg qhs (IR Tablet) | | | | | |
| Treatment B: Rifaximin SSD | 55 | 11 (20.0%) | 7 (12.7%) | 37 (67.3%) | 0.2297 |
| 80 mg qhs (IR Tablet) | | | | | |
| Treatment C: Rifaximin SSD | 53 | 4 (7.5%) | 5 (9.4%) | 44 (83.0%) | 0.4007 |
| 40 mg qhs (SER Tablet) | | | | | |
| Treatment D: Rifaximin SSD | 48 | 1 (2.1%) | 4 (8.3%) | 43 (89.6%) | 0.0460 |
| 80 mg qhs (SER Tablet) | | | | | |
| Treatment E: Rifaximin SSD | 56 | 11 (19.6%) | 1 (1.8%) | 44 (78.6%) | 0.3340 |
| 80 mg qhs (IR Tablet) and | | | | | |
| Rifaximin SSD 80 mg qhs | | | | | |
| (SER Tablet) | | | | | |
| Conn Score: 1 | | | | | |
| Overall Treatment Comparison³ | | | | | |
| Any Rifaximin SSD Treatment | 160 | 15 (9.4%) | 11 (6.9%) | 134 (83.8%) | 0.8251 |
| Placebo | 37 | 3 (8.1%) | 5 (13.5%) | 29 (78.4%) | |
| Pairwise Comparisons | | | | | |
| (versus Placebo)³ | | | | | |
| Treatment A: Rifaximin SSD | 30 | 0 | 2 (6.7%) | 28 (93.3%) | 0.0941 |
| 40 mg qhs (IR Tablet) | | | | | |
| Treatment B: Rifaximin SSD | 36 | 4 (11.1%) | 2 (5.6%) | 30 (83.3%) | 0.7015 |
| 80 mg qhs (IR Tablet) | | | | | |
| Conn Score 1 Pairwise Comparisons | | | | | |
| (versus Placebo)³, Continued | | | | | |
| Treatment C: Rifaximin SSD | 31 | 5 (16.1%) | 2 (6.5%) | 24 (77.4%) | 0.3467 |
| 40 mg qhs (SER Tablet) | | | | | |
| Treatment D: Rifaximin SSD | 39 | 2 (5.1%) | 2 (5.1%) | 35 (89.7%) | 0.5204 |
| 80 mg qhs (SER Tablet) | | | | | |
| Treatment E: Rifaximin SSD | 24 | 4 (16.7%) | 3 (12.5%) | 17 (70.8%) | 0.3075 |
| 80 mg qhs (IR Tablet) and | | | | | |
| Rifaximin SSD 80 mg qhs (SER | | | | | |
| Tablet) | | | | | |
| Conn Score: 2 | | | | | |
| Overall Treatment Comparison³ | | | | | |
| Any Rifaximin SSD Treatment | 2 | 1 (50.0%) | 0 | 1 (50.0%) | |
| Pairwise Comparisons | | | | | |
| (versus Placebo)³ | | | | | |
| Treatment D: Rifaximin SSD | 2 | 1 (50.0%) | 0 | 1 (50.0%) | |
| 80 mg qhs (SER Tablet) | | | | | |

IR = immediate release; ITT = intent to treat; qhs = once daily at bedtime; SER = sustained extended release; SSD = solid soluble dispersion.
¹Number of subjects censored at Week 24 (subject did not experience an event and was enrolled in the study at Week 24).
²P-value was obtained using a stratified log-rank test.
³Stratified by analysis region (study centers are grouped within 2 regions, centers in the United States and centers in Russia)

### Time Since First Diagnosis of Liver Cirrhosis

The influence of a subject's time since first diagnosis of liver cirrhosis on the primary efficacy analysis was evaluated. The time since first diagnosis of liver cirrhosis subgroups were categorized as <947 days or ≥947 days. Table 7 presents the analysis of the primary efficacy endpoint by time since first diagnoses of liver cirrhosis. A near statistically significant difference in the time to hospitalization for any of the liver cirrhosis complications or all-cause mortality was observed within ≥947 days subgroup and, like the PP and baseline Conn score 0 populations, was in favor of the SER 80 mg qhs treatment group versus placebo (stratified log-rank p=0.0517). The overall treatment comparison effect for any of the rifaximin SSD treatment versus placebo was not statistically significant (time since first diagnosis of liver cirrhosis: <947 days stratified log-rank p=0.3961; time since first diagnosis of liver cirrhosis: ≥947 days stratified log-rank p=0.5689).

TABLE 7
Analysis of Primary Efficacy Endpoint: Time to Hospitalization for any of the
Liver Cirrhosis Complications or All-cause Mortality by Categorized Time Since
First Diagnosis of Liver Cirrhosis up to 24 Weeks (Day 170) - ITT Population

| | | | Censored | | |
|---|---|---|---|---|---|
| | # of Subjects | # of Events | <Week 24 | Week 24¹ | p-value² |
| <947 Days | | | | | |
| Overall Treatment Comparison³ | | | | | |
| Any Rifaximin SSD Treatment | 206 | 32 (15.5%) | 14 (6.8%) | 160 (77.7%) | 0.3961 |
| Placebo | 50 | 5 (10.0%) | 7 (14.0%) | 38 (76.0%) | |
| Pairwise Comparisons | | | | | |
| (versus Placebo)³ | | | | | |
| Treatment A: Rifaximin SSD | 43 | 3 (7.0%) | 3 (7.0%) | 37 (86.0%) | 0.4929 |
| 40 mg qhs (IR Tablet) | | | | | |
| Treatment B: Rifaximin SSD | 41 | 8 (19.5%) | 7 (17.1%) | 26 (63.4%) | 0.2329 |
| 80 mg qhs (IR Tablet) | | | | | |
| Treatment C: Rifaximin SSD | 46 | 8 (17.4%) | 2 (4.3%) | 36 (78.3%) | 0.3436 |
| 40 mg qhs (SER Tablet) | | | | | |
| Treatment D: Rifaximin SSD | 34 | 3 (8.8%) | 1 (2.9%) | 30 (88.2%) | 0.7582 |
| 80 mg qhs (SER Tablet) | | | | | |

| | Censored | | | Censored | |
|---|---|---|---|---|---|
| | # of Subjects | # of Events | | # of Subjects | # of Events |
| ≥947 Days | | | | | |
| Overall Treatment Comparison³ | | | | | |
| Any Rifaximin SSD Treatment | 215 | 18 (8.4%) | 16 (7.4%) | 181 (84.2%) | 0.5689 |
| Placebo | 44 | 5 (11.4%) | 4 (9.1%) | 35 (79.5%) | |
| Pairwise Comparisons | | | | | |
| (versus Placebo)³ | | | | | |
| Treatment A: Rifaximin SSD | 34 | 4 (11.8%) | 1 (2.9%) | 29 (85.3%) | 0.9598 |
| 40 mg qhs (IR Tablet) | | | | | |
| Treatment B: Rifaximin SSD | 50 | 7 (14.0%) | 2 (4.0%) | 41 (82.0%) | 0.6094 |
| 80 mg qhs (IR Tablet) | | | | | |
| Treatment C: Rifaximin SSD | 38 | 1 (2.6%) | 5 (13.2%) | 32 (84.2%) | 0.1523 |
| 40 mg qhs (SER Tablet) | | | | | |
| Treatment D: Rifaximin SSD | 55 | 1 (1.8%) | 5 (9.1%) | 49 (89.1%) | 0.0517 |
| 80 mg qhs (SER Tablet) | | | | | |
| Treatment E: Rifaximin SSD | 38 | 5 (13.2%) | 3 (7.9%) | 30 (78.9%) | 0.8519 |
| 80 mg qhs (IR Tablet) and | | | | | |
| Rifaximin SSD 80 mg qhs | | | | | |
| (SER Tablet) | | | | | |

IR = immediate release; ITT = intent to treat; qhs = once daily at bedtime; SER = sustained extended release; SSD = solid soluble dispersion.
¹Number of subjects censored at Week 24 (subject did not experience an event and was enrolled in the study at Week 24).
²P-value was obtained using a stratified log-rank test.
³Stratified by analysis region (study centers are grouped within 2 regions, centers in the United States and centers in Russia).

### Time to Development of Medically Refractory Ascites up to Week 24 (Day 170)

Analysis of the time to development of medically refractory ascites up to Week 24 (Day 170) is presented in Table 8.

A statistically significant difference in time to development of medically refractory ascites up to 24 Week was observed in favor of the IR 40 mg qhs treatment group versus placebo (stratified log-rank p=0.0308) and in favor of the SER 40 mg qhs treatment group versus placebo (stratified log-rank p=0.0202). No other pairwise treatment comparisons versus placebo were statistically significant. The overall treatment comparison for any of the rifaximin SSD treatments versus placebo was not statistically significant.

TABLE 8
Analysis of Secondary Efficacy Endpoint: Time to Development of Medically
Refractory Ascites up to 24 Week (Day 170) - ITT Population

| | | | Censored | | |
|---|---|---|---|---|---|
| | # of Subjects | # of Events | <Week 24 | Week 24¹ | p-value² |
| Overall Treatment | | | | | |
| Comparison³ | | | | | |
| Any Rifaximin SSD | 422 | 16 (3.8%) | 51 (12.1%) | 355 (84.1%) | 0.0601 |
| Treatment | | | | | |
| Placebo | 94 | 0 | 13 (13.8%) | 81 (86.2%) | |
| Pairwise Comparisons | | | | | |
| (versus Placebo)³ | | | | | |
| Treatment A: Rifaximin | 78 | 4 (5.1%) | 8 (10.3%) | 66 (84.6%) | 0.0308 |
| SSD 40 mg qhs | | | | | |
| (Immediate Release [IR] | | | | | |
| Tablet) | | | | | |
| Treatment B: Rifaximin | 91 | 3 (3.3%) | 18 (19.8%) | 70 (76.9%) | 0.0721 |
| SSD 80 mg qhs | | | | | |
| (Immediate Release [IR] | | | | | |
| Tablet) | | | | | |
| Treatment C: Rifaximin | 84 | 5 (6.0%) | 9 (10.7%) | 70 (83.3%) | 0.0202 |
| SSD 40 mg qhs | | | | | |
| (Sustained Extended | | | | | |
| Release [SER] Tablet) | | | | | |
| Treatment D: Rifaximin | 89 | 2 (2.2%) | 8 (9.0%) | 79 (88.8%) | 0.1508 |
| SSD 80 mg qhs | | | | | |
| (Sustained Extended | | | | | |
| Release [SER] Tablet) | | | | | |
| Treatment E: Rifaximin | 80 | 2 (2.5%) | 8 (10.0%) | 70 (87.5%) | 0.1319 |
| SSD 80 mg qhs | | | | | |
| (IR Tablet) and Rifaximin | | | | | |
| SSD 80 mg qhs (SER | | | | | |
| Tablet) | | | | | |

IR = immediate release; ITT = intent to treat; qhs = once daily at bedtime; SER = sustained extended release; SSD = solid soluble dispersion.
¹Number of subjects censored at Week 24 (subject did not experience an event and was enrolled in the study at Week 24).
²P-value was obtained using a stratified log-rank test.
³Stratified by analysis region (study centers are grouped within 2 regions, centers in the United States and centers in Russia).

### Efficacy Conclusions

Based on Kaplan Meier estimates of distribution of time to hospitalization for any of the liver cirrhosis complications or all-cause mortality up to 24 weeks, there was a statistically significant effect in favor of the SER 80 mg qhs and combined IR/SER qhs treatment groups having the highest and lowest survival rates, respectively.

The primary analysis on the PP population did demonstrate a statistically significant difference in the time to hospitalization for any of the liver cirrhosis complications or all-cause mortality up to 24 weeks that was in favor of the SER 80 mg qhs treatment group versus placebo. Kaplan Meier estimates of distribution of time to hospitalization for any of the liver cirrhosis complications or all-cause mortality up to 24 weeks were also statistically significant in favor of the SER 80 mg qhs and combined IR/SER qhs treatment groups having the highest and lowest survival rates, respectively.

In the secondary analysis, there was a statistically significant difference in time to development of medical refractory ascites up to Week 24 in favor of the IR 40 mg qhs and SER 40 mg qhs treatment groups versus placebo. There was a statistically significant effect for change from baseline in ESS total score was statistically significant treatment versus placebo effect was observed at Week 4 at the 25th percentile for baseline (p<0.0001), with the IR 40 mg qhs treatment group presenting with the greatest decrease from baseline.

These studies show, for the primary analysis, overall time to hospitalization for any of the liver cirrhosis complications or all-cause mortality up to 24 weeks was in favor of the SER 80 mg qhs treatment group versus placebo. In the secondary analysis, statistically significant favorable effects were observed most consistently in the IR 40 mg qhs treatment group as well as occurrences in the combined IR/SER qhs and SER 40 mg qhs treatment groups.

### Example 2. Use of Rifaximin SSD IR 40 mg for the Treatment of Overt Hepatic Encephalopathy

Rifaximin SSD as described in Table 3 was studied in a randomized, double-blind, placebo-controlled, dose-ranging, multicenter study to assess the efficacy and safety of Rifaximin Soluble Solid Dispersion (SSD) Tablets Plus lactulose for the treatment of Overt Hepatic Encephalopathy (OHE).

The primary objective of this study was to assess the efficacy of rifaximin SSD plus lactulose versus placebo plus lactulose for the treatment of overt hepatic encephalopathy (OHE). The adult, oral dosage of lactulose is 2 to 3 tablespoons (30 to 45 mL, containing 20 g to 30 g of lactulose) three times daily. The dosage was adjusted to produce 2 to 3 soft stools daily or per PI discretion. The secondary objectives of this study were to assess the safety of rifaximin SSD in subjects with OHE and to assess the effects of treatment with rifaximin SSD on key secondary endpoints. The primary endpoint was the time to OHE resolution, determined using the Hepatic Encephalopathy Grading Instrument (HEGI). Secondary endpoints included: time to OHE resolution defined as a West Haven Criteria (WHC) score of 0 or 1, time to 1 unit decrease in WHC score, change from baseline in HEGI score at Day 14 or time of discharge (end of randomized treatment period), whichever occurs first, time to improvement in HEGI score, change from baseline in WHC score at Day 14 or time of discharge (end of randomized treatment period), whichever occurs first, and length of hospitalization from baseline to discharge.

Results

A total of 71 subjects completed the trial: 15 subjects in the rifaximin 40 mg IR QD arm, 15 subjects in the rifaximin 40 mg IR BID arm, 14 subjects in the 80 mg SER QD arm, 13 subjects in the 80 mg SER BID arm, and 14 subjects in the PBO arm. Demographics and baseline characteristics were similar across the treatment groups. The average age was 61. Fifty four percent of subjects were male. Seventy percent of subjects entered the trial with a HEGI score of 2, 30% entered with a HEGI score of 3. The mean MELD score on admission to the trial was 21.2. The mean number of HE episodes in the six months prior to trial admission was 1.2.

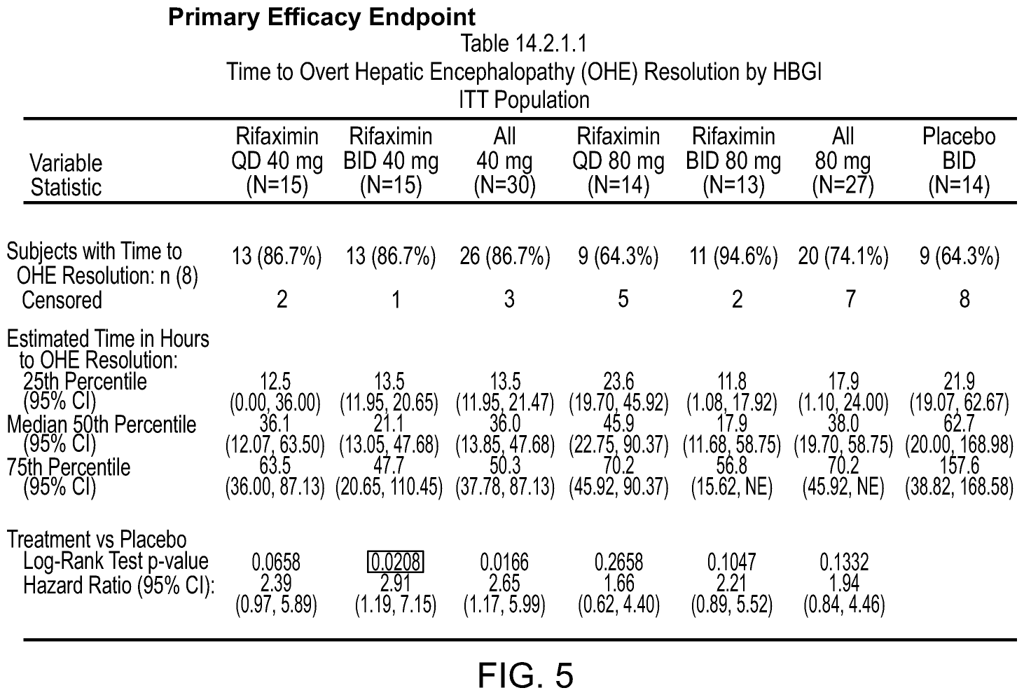

Primary endpoint—Time to OHE resolution determined using the Hepatic Encephalopathy Grading Instrument (HEGI). The Rifaximin BID 40 mg arm was statistically significantly superior to the placebo arm (P=0.0208). For the Rifaximin BID 40 mg arm, the median time to resolution was 21.1 hours vs. 62.7 hours for the placebo arm. A subject's probability of achieving resolution at any given time was 2.91 times higher for subjects in this arm than for subjects in the placebo arm. The 40 mg QD arm trended towards statistical significance with a p value of 0.0658. Although not statistically significant, both 80 mg SER arms demonstrated general numerical superiority over PBO regarding the primary endpoint (FIG. 5).

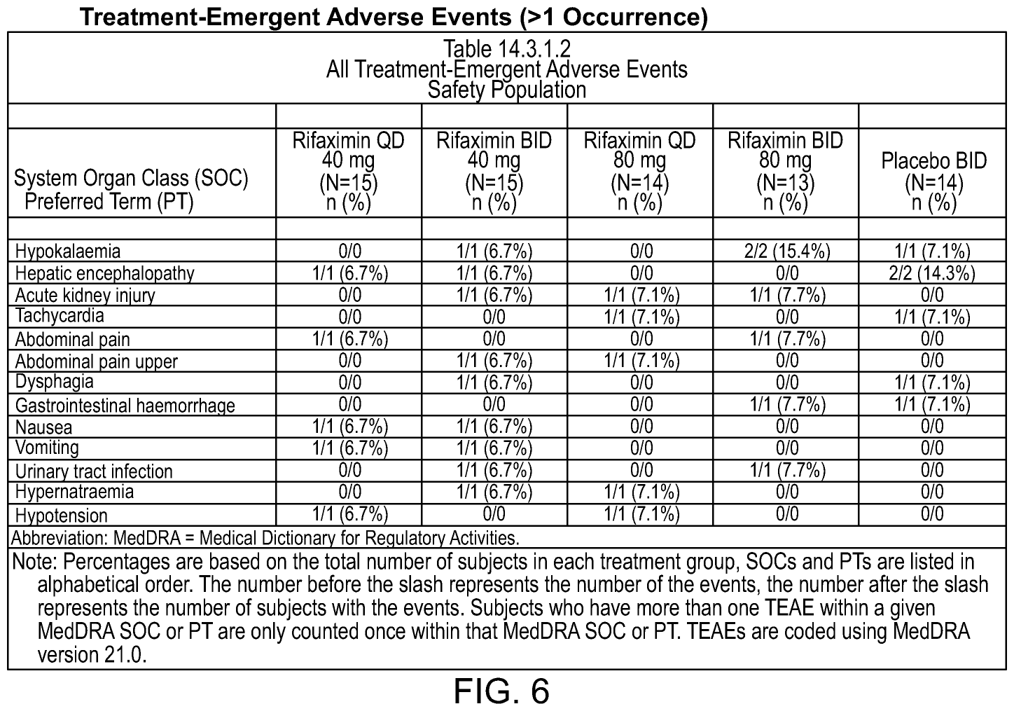

Secondary endpoints—The rifaximin arms were not statistically significantly different from the placebo arm regarding time to OHE resolution by WHC, but demonstrated numerical superiority across the board. The rifaximin arms were not statistically significantly different from the placebo arm regarding time to a one unit decrease in WHC score but demonstrated numerical superiority across the board, especially the 40 IR QD arm. Regarding time to change in HEGI score, The Rifaximin BID 40 mg arm was statistically significantly superior to the placebo arm (P=0.0379). In the Rifaximin BID 40 mg arm, the median time to change in HEGI score was 2.0 days vs. 3.0 days for the placebo arm. A subject's probability of achieving a change in HEGI score at any given time was 1.94 times higher for subjects in this arm than for subjects in the placebo arm. No safety issues were identified; the rates of adverse events and serious AEs were similar across the treatment groups (FIG. 6).

### CONCLUSION

The rifaximin containing SSD IR tablets, delivered BID, at a 40 mg per tablet dosage, were found to treat patients having OHE when combined with lactulose when compared to lactulose alone. Indeed, as shown above, patients treated with the foregoing method achieved OHE resolution at a rate that was 2.91 times higher than for subjects receiving lactulose alone as monitored by HEGI. Furthermore, patients subjected to this method were almost twice as likely (i.e., 1.94 times) to achieve a change in HEGI score as compared to those receiving lactulose alone.

### Example 3. Use of Rifaximin SSD IR 40 mg for Delaying the Onset of Overt Hepatic Encephalopathy (OHE) in Patients with Liver Decompensation

40 mg SSD IR as described in Table 3 will be investigated in a Phase 3, randomized, double-blind, placebo-controlled, multicenter study to assess its efficacy and safety for the delay of the first episode of overt hepatic encephalopathy decompensation in advanced cirrhosis, defined by the presence of medically controlled ascites. Subjects who have cirrhosis determined by histopathological evidence, transient elastography or presence of esophageal varices, and who have not previously experienced OHE, spontaneous bacterial peritonitis (SBP), esophageal variceal bleeding (EVB), or hepatorenal syndrome (HRS) will be enrolled in the study. Subjects will complete a 28-day Screening Period, a 72-week Treatment Period, and a 4-week Follow-up Period. Subjects who successfully complete the Screening Period will enter the Treatment Period and will be randomized in a 1:1 allocation to one of two treatment groups as noted above. All treatments will be administered twice daily (BID).

The use of rifaximin in preventing complications of cirrhosis is supported by multiple lines of clinical and experimental evidence. For example, and in addition to the evidence described herein, rifaximin was approved by the US FDA in March 2010 for the reduction in risk of recurrent overt HE; is shown to protect from HE recurrences with decreased HE-related and all-cause hospitalizations without an increased rate of adverse events (AEs) or decreased survival (see e.g., Conf. Proc. IEEE Eng. Med. Biol. Soc. 2013 2184-2187); reduces or maintains the overall rates of infection, antibiotic use, and other complications of cirrhosis such as ascites (see Conf. Proc. IEEE Eng. Med. Biol. Soc. supra); and was independently associated with higher survival and lower risk of developing variceal bleeding, HE, SBP, or HRS (see e.g., Journal of Gastroenterology and Hepatology 28(3); December 2012).

The subject population includes male and non-pregnant, non-nursing females ≥18 years with liver cirrhosis and medically controlled ascites who have not previously experienced an episode of OHE, SBP, EVB or HRS.

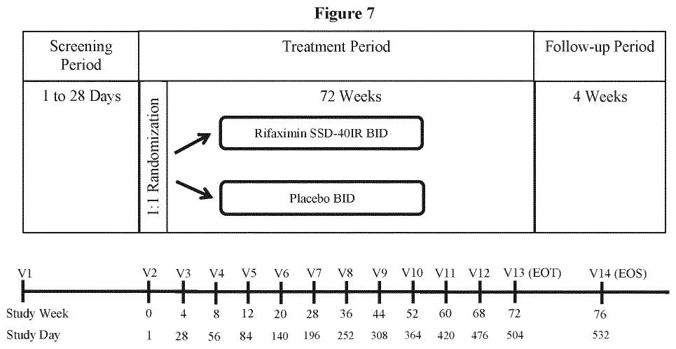

Assessments of efficacy and safety will be performed during clinic visits at Weeks 0 (Day 1), 4, 8, 12, 20, 28, 36, 44, 52, 60, 68 and 72 (EOT) and during telephone calls at Weeks 16, 24, 32, 40, 48, 56 and 64. All subjects will complete an End of Study (EOS) visit at Week 76 for final safety assessments. If a subject prematurely discontinues treatment, every attempt will be made to obtain EOT and EOS assessments. A representative study design is shown by FIG. 7.

A subject will be eligible for inclusion in this study if he/she meets all of the following criteria:
1. Subject has a diagnosis of advanced liver cirrhosis with medically controlled ascites (>30 days) not requiring paracentesis (prophylactic variceal banding allowed if no history of previous variceal bleeding).
   Cirrhosis diagnosis can be made using any of the following:
      Histopathological evidence of cirrhosis
      Imaging (sonographic or cross-sectional)
      Fibroscan (Transient Elastography)
      Presence of esophageal varices
      Thrombocytopenia (<150,000) in CLD
2. Subject has a Child-Pugh B Classification (score of 7 to 9 inclusive) without OHE and a Model End Stage Liver Disease-Sodium (MELD-Na) score of <15 at Visit one.
3. Subject has a Conn (West Haven Criteria) score of <2.
4. Subject has a Mini-Mental State Examination (MMSE) score ≥25 and demonstrates no cognitive impairment upon exam.
   5. Subject is greater than or equal to 18 years of age.
   6. Females of childbearing (reproductive) potential must have a negative serum or urine pregnancy test at screening and agree to use an acceptable method of contraception throughout their participation in the study.
   7. Subject must be able to read, understand and provide written informed consent on the Institutional Review Board (IRB)/Ethics Committee (EC) approved informed consent form (ICF) and provide authorization as appropriate per local privacy regulations.

A subject will not be eligible for inclusion in this study if any of the following criteria apply:
1. Subject has a history of hypersensitivity to rifaximin, rifampin, rifamycin antimicrobial agents, or any of the components of rifaximin.
2. Subject has a history of SBP, primary or secondary SBP prophylaxis, EVB, or HRS.
3. Subject has a documented history of an OHE episode (Conn score ≥2) or has a history of rifaximin 550 mg and lactulose use for suspected OHE episode.
4. Subject has either: a) no ascites -or- b) uncontrolled ascites.
5. Subject has other non-controllable neurological or psychiatric conditions which may confound the assessment of cognitive function (e.g., dementia, schizophrenia, etc.).
6. Subject has signs of alcohol use disorder, defined as AUDIT-10 score of >7, within 6 weeks of signing the ICF.
7. Subject has a history of substance abuse <6 weeks prior to signing the informed consent form and cannot refrain from substance abuse during the study period.
8. Subject has been diagnosed with an uncontrolled infection <4 weeks prior to screening.
9. Subject has been diagnosed with an upper gastrointestinal bleed from non-variceal sources <6 weeks prior to screening.
10. Subject shows the presence of intestinal obstruction or has inflammatory bowel disease.
11. Subject has a history of shunt surgery or a transjugular intrahepatic portosystemic shunt (TIPS) procedure for portal hypertension.
12. Subject has undergone prophylactic variceal banding within 2 weeks of screening or is scheduled to undergo prophylactic variceal banding during the study
(Note: subjects with previous prophylactic variceal banding will be allowed to participate in the study).
13. Subject has Type 1 or Type 2 diabetes that is not adequately controlled in the opinion of the investigator.
14. Subject has severe co-morbid disease with a life expectancy <1 year.
15. Subject has active malignancy, including active hepatocellular carcinoma (HCC).
16. Subject requires hemodialysis.
17. Subject has any condition or circumstance that adversely affects the subject, could cause noncompliance with treatment or visits, may impact the interpretation of clinical data, could cause bias, or may otherwise contraindicate the subject's participation in the study.
18. Subject used any investigational product or device within 30 days of providing consent.
19. Subjects with focal neurological deficits due to a neurological event such as cerebrovascular accident.
20. Subject consumes more than moderate amounts of alcohol, defined as one standard drink per day for women and two standard drinks per day for men.
21. Subject is currently taking narcotics, benzodiazepines, or psychoactive medicines which cannot be discontinued.
22. Subject has undergone bariatric surgery or intestinal resection.

Efficacy Endpoints

Primary efficacy endpoint will include the time to first event of OHE requiring hospitalization or emergency department visit with initiation of HE specific therapies (lactulose, IV hydration, etc.). Alternatively, the efficacy endpoint may be defined as the time to first event of OHE as diagnosed by a physician that requires a referral to an emergency department with initiation of HE specific therapies.

Secondary efficacy endpoint will include time to all-cause hospitalization.

Exploratory endpoints will include 1) Time to first non-OHE complication of cirrhosis, defined as any of the following events: SBP, Variceal bleed, HRS; 2) Change from baseline in MELD and MELD-Na score; 3) Change from baseline in visuo-spatial functioning, attention and processing speed using psychometric hepatic encephalopathy scores (PHES); 4) Change from baseline in health-related quality of life (HRQL) according to the Patient-Reported Outcomes Measurement Information System (PROMIS) Computer Adaptive Test (CAT) and the 36-Item Short Form (SF-36) Health Survey; 5) Change from baseline in Caregiver Reported Burden; 6) Time to one point increase from baseline in Conn score; 7) Population pharmacokinetics; and 8) microbiota and metabolome analysis for at least a subset of patients.

### NUMBERED EMBODIMENTS

Embodiment 1a. A method of treating Overt Hepatic Encephalopathy (OHE) in a patient in need thereof comprising administering at least one 40 mg rifaximin SSD IR tablet to the patient until OHE symptoms resolve.

Embodiment 2a. A method of treating Overt Hepatic Encephalopathy (OHE) in a patient in need thereof comprising administering at least one 40 mg rifaximin SSD IR tablet to the patient for at least one day, or at least two days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days.

Embodiment 3a. A method of treating Overt Hepatic Encephalopathy (OHE) in a patient in need thereof comprising administering a 40 mg rifaximin SSD IR tablet QD to the patient for at least one day, or at least two days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days.

Embodiment 4a. A method of treating Overt Hepatic Encephalopathy (OHE) in a patient in need thereof comprising administering a 40 mg rifaximin SSD IR tablet BID to the patient for at least one day, or at least two days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days.

Embodiment 5a. A method of treating Overt Hepatic Encephalopathy (OHE) in a patient in need thereof comprising administering a 40 mg rifaximin SSD IR tablet TID to the patient for at least one day, or at least two days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days.

Embodiment 6a. A method of treating Overt Hepatic Encephalopathy (OHE) in a patient in need thereof comprising administering at least one 40 mg rifaximin SSD IR tablet to the patient until the patient's Hepatic Encephalopathy Grading Instrument (REGI) score or West Haven Criteria (WHC) score is reduced by at least one grade, or at least two grades, or at least three grades.

Embodiment 7a. A method of treating Overt Hepatic Encephalopathy (OHE) in a patient in need thereof comprising administering at least one 40 mg rifaximin SSD IR tablet to the patient until the patient's Hepatic Encephalopathy Grading Instrument (HEGI) score is reduced from grade 4 to grade 3 or from grade 3 to grade 2.

Embodiment 8a. A method of treating Overt Hepatic Encephalopathy (OHE) in a patient in need thereof comprising administering a 40 mg rifaximin SSD IR tablet QD to the patient until the patient's Hepatic Encephalopathy Grading Instrument (HEGI) score or West Haven Criteria (WHC) score is reduced by at least one grade, or at least two grades, or at least three grades.

Embodiment 9a. A method of treating Overt Hepatic Encephalopathy (OHE) in a patient in need thereof comprising administering a 40 mg rifaximin SSD IR tablet QD to the patient until the patient's Hepatic Encephalopathy Grading Instrument (HEGI) score or West Haven Criteria (WHC) score is reduced from grade 4 to grade 3 or from grade 3 to grade 2 or from grade 2 to grade 1.

Embodiment 10a. A method of treating Overt Hepatic Encephalopathy (OHE) in a patient in need thereof comprising administering a 40 mg rifaximin SSD IR tablet BID to the patient until the patient's Hepatic Encephalopathy Grading Instrument (HEGI) score or West Haven Criteria (WHC) score is reduced by at least one grade, or at least two grades, or at least three grades.

Embodiment 11a. A method of treating Overt Hepatic Encephalopathy (OHE) in a patient in need thereof comprising administering a 40 mg rifaximin SSD IR tablet BID to the patient until the patient's Hepatic Encephalopathy Grading Instrument (HEGI) score or West Haven Criteria (WHC) score is reduced from grade 4 to grade 3 or from grade 3 to grade 2 or from grade 2 to grade 1.

Embodiment 12a. A method of treating Overt Hepatic Encephalopathy (OHE) in a patient in need thereof comprising administering a 40 mg rifaximin SSD IR tablet TID to the patient until the patient's Hepatic Encephalopathy Grading Instrument (HEGI) score or West Haven Criteria (WHC) score is reduced by at least one grade, or at least two grades, or at least three grades.

Embodiment 13a. A method of treating Overt Hepatic Encephalopathy (OHE) in a patient in need thereof comprising administering a 40 mg rifaximin SSD IR tablet TID to the patient until the patient's Hepatic Encephalopathy Grading Instrument (HEGI) score or West Haven Criteria (WHC) score is reduced from grade 4 to grade 3 or from grade 3 to grade 2 or from grade 2 to grade 1.

Embodiment 14a. The method of any one of Embodiments 1a to 13a, wherein the method further comprises administering lactulose to the patient.

Embodiment 15a. The method of embodiment 14a, wherein the lactulose and rifaximin are administered concomitantly.

Embodiment 16a. The method of embodiment 14a, wherein the lactulose and rifaximin are administered sequentially.

Embodiment 17a. A method for treating Overt Hepatic Encephalopathy (OHE) in a patient in need thereof as described herein.

Embodiment 18a. A method of treating Overt Hepatic Encephalopathy (OHE) in a patient in need thereof comprising administering to the patient at least one rifaximin SSD IR tablet.

Embodiment 19a. The method of embodiment 18a, wherein the at least one rifaximin SSD IR tablet is administered until the patient's Hepatic Encephalopathy Grading Instrument (REGI) score or West Haven Criteria (WHC) score is reduced by at least one grade, or at least two grades, or at least three grades.

Embodiment 20a. The method of embodiment 18a or 19a, wherein the at least one rifaximin SSD IR tablet is administered until the patient's Hepatic Encephalopathy Grading Instrument (REGI) score or West Haven Criteria (WHC) score is reduced from grade 4 to grade 3 or from grade 3 to grade 2 or from grade 2 to grade 1.

Embodiment 21a. A method of preventing complications of liver cirrhosis or all-cause mortality, or both, in a patient with liver decompensation comprising administering to the patient at least one rifaximin SSD IR tablet.

Embodiment 22a. The method of embodiment 21a, wherein the complications of liver cirrhosis comprise one or more of hepatic encephalopathy (HE), esophageal variceal bleeding (EVB), spontaneous bacterial peritonitis (SBP), and hepatorenal syndrome (HRS).

Embodiment 23a. The method of embodiment 21a or 22a, wherein the patient has documented medically controlled non-refractory ascites.

Embodiment 24a. The method of any one of embodiments 21a to 23a, wherein the patient has not previously experienced spontaneous bacterial peritonitis (SBP), esophageal variceal bleeding (EVB), or hepatorenal syndrome (HRS) prior to treatment.

Embodiment 25a. The method of any one of embodiment 21a to 24a, wherein said method further comprises reducing the risk of hospitalizations associated with complications of liver cirrhosis or all-cause mortality, or both.

Embodiment 26a. A method of delaying the onset of overt hepatic encephalopathy (OHE) in patients with liver decompensation comprising administering to the patient at least one rifaximin SSD IR tablet.

Embodiment 27a. The method of embodiment 26, wherein liver decompensation comprises cirrhosis of the liver and medically controlled ascites.

Embodiment 28a. The method of embodiment 27a, wherein the ascites are medically controlled for a period of at least 30 days.

Embodiment 29a. The method of embodiment 27a or 28a, wherein cirrhosis of the liver is determined by one or more of histopathological evidence of cirrhosis, transient elastography, the presence of esophageal varices, and thrombocytopenia characterized by a blood platelet level of less than 150,000 in a patient with chronic liver disease.

Embodiment 30a. The method of any one of embodiments 26a to 29a, wherein the patient has a Child-Pugh B Classification score of from 7 to 9 without OHE prior to treatment.

Embodiment 31a. The method of any one of embodiments 26a to 30a, wherein the patient has a Model End Stage Liver Disease-Sodium (MELD-Na) score of less than 15 prior to treatment.

Embodiment 32a. The method of any one of embodiments 26a to 31a, wherein the patient has a Conn (West Haven Criteria) score of less than 2 prior to treatment.

Embodiment 33a. The method of any one of embodiments 26a to 32a, wherein the patient has a Mini-Mental State Examination (MMSE) score of at least 25 prior to treatment.

Embodiment 34a. The method of any one of embodiments 26a to 33a, wherein the patient demonstrates no cognitive impairment prior to treatment.

Embodiment 35a. The method of any one of embodiments 26a to 34a, wherein OHE comprises a Conn score of 2 or higher.

Embodiment 36a. The method of any one of embodiments 26a to 35a, wherein the patient does not require paracentesis.

Embodiment 37a. The method of any one of embodiments 18a to 36a, wherein the at least one rifaximin SSD IR tablet comprises about 40 mg of rifaximin.

Embodiment 38a. The method of any one of embodiments 18a to 37a, wherein the at least one rifaximin SSD IR tablet is administered to the patient for at least one day, or at least two days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days.

Embodiment 39a. The method of any one of embodiments 18a to 38a, wherein the at least one rifaximin SSD IR tablet is administered to the patient once a day, twice a day, or three times a day.

Embodiment 40a. The method of any one of embodiments 18a to 39a, wherein the method further comprises administering lactulose to the patient.

Embodiment 41a. The method of embodiment 40a, wherein the lactulose and rifaximin are administered concomitantly.

Embodiment 42a. The method of embodiment 41a, wherein the lactulose and rifaximin are administered sequentially.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

### FURTHER NUMBERED EMBODIMENTS

Embodiment 1b. A rifaximin SSD IR tablet comprising about 40 mg of rifaximin, for use in a method of treating Overt Hepatic Encephalopathy (OHE) in a patient in need thereof.

According to a preferred embodiment at least one rifaximin SSD IR tablet is administering to the patient.

Embodiment 2b. The tablet for use according to embodiment 1b, wherein the tablet is administered until the patient's Hepatic Encephalopathy Grading Instrument (HEGI) score or West Haven Criteria (WHC) score is reduced by at least one grade, or at least two grades, or at least three grades.

Embodiment 3b. The tablet for use according to embodiment 1b or 2b, wherein the tablet is administered until the patient's Hepatic Encephalopathy Grading Instrument (HEGI) score or West Haven Criteria (WHC) score is reduced from grade 4 to grade 3 or from grade 3 to grade 2 or from grade 2 to grade 1.

Embodiment 4b. A rifaximin SSD IR tablet comprising about 40 mg of rifaximin, for use in a method of preventing complications of liver cirrhosis or all-cause mortality, or both, in a patient with liver decompensation.

According to a preferred embodiment at least one rifaximin SSD IR tablet is administering to the patient.

Embodiment 5b. The tablet for use according to embodiment 4b, wherein the complications of liver cirrhosis comprise one or more of hepatic encephalopathy (HE), esophageal variceal bleeding (EVB), spontaneous bacterial peritonitis (SBP), and hepatorenal syndrome (HRS).

Embodiment 6b. The tablet for use according to embodiment 4b or 5b, wherein the patient has documented medically controlled non-refractory ascites.

Embodiment 7b. The tablet for use according to any one of embodiments 4b to 6b, wherein the patient has not previously experienced spontaneous bacterial peritonitis (SBP), esophageal variceal bleeding (EVB), or hepatorenal syndrome (HRS) prior to treatment.

Embodiment 8b. The tablet for use according to any one of embodiments 4b to 7b, wherein the tablet is further used in a method of reducing the risk of hospitalizations associated with complications of liver cirrhosis or all-cause mortality, or both.

Embodiment 9b. A rifaximin SSD IR tablet comprising about 40 mg of rifaximin, for use in a method of delaying the onset of overt hepatic encephalopathy (OHE) in patients with liver decompensation.

According to a preferred embodiment at least one rifaximin SSD IR tablet is administering to the patient.

Embodiment 10b. The tablet for use according to embodiment 9b, wherein the liver decompensation comprises cirrhosis of the liver and medically controlled ascites.

Embodiment 11b. The tablet for use according to embodiment 10b, wherein the ascites are medically controlled for a period of at least 30 days.

Embodiment 12b. The tablet for use according to embodiment 10b or 11b, wherein cirrhosis of the liver is determined by one or more of histopathological evidence of cirrhosis, transient elastography, the presence of esophageal varices, and thrombocytopenia characterized by a blood platelet level of less than 150,000 in a patient with chronic liver disease.

Embodiment 13b. The tablet for use according to embodiments 10b to 12b, wherein the patient has a Child-Pugh B Classification score of from 7 to 9 without OHE prior to treatment.

Embodiment 14b. The tablet for use according to embodiments 10b to 13b, wherein the patient has a Model End Stage Liver Disease-Sodium (MELD-Na) score of less than 15 prior to treatment.

Embodiment 15b. The tablet for use according to embodiments 10b to 14b, wherein the patient has a Conn (West Haven Criteria) score of less than 2 prior to treatment.

Embodiment 16b. The tablet for use according to embodiments 10b to 15b, wherein OHE comprises a Conn score of 2 or higher.

Embodiment 17b. The tablet for use according to embodiments 9b to 16b, wherein the patient does not require paracentesis.

Embodiment 18b. The tablet for use according to embodiments 1b to 17b, wherein the tablet is administered to the patient for at least one day, or at least two days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days.

Embodiment 19b. The tablet for use according to embodiments 1b to 18b, wherein the tablet is administered to the patient once a day, twice a day, or three times a day.

Embodiment 20b. The tablet for use according to embodiments 1b to 19b, wherein further to the tablet lactulose is administered to the patient.

1. A method of treating Overt Hepatic Encephalopathy (OHE) in a patient in need thereof comprising administering to the patient at least one rifaximin SSD IR tablet comprising about 40 mg of rifaximin.

2. The method of claim 1, wherein the at least one rifaximin SSD IR tablet is administered until the patient's Hepatic Encephalopathy Grading Instrument (HEGI) score or West Haven Criteria (WHC) score is reduced by at least one grade, or at least two grades, or at least three grades.

3. The method of claim 1, wherein the at least one rifaximin SSD IR tablet is administered until the patient's Hepatic Encephalopathy Grading Instrument (HEGI) score or West Haven Criteria (WHC) score is reduced from grade 4 to grade 3 or from grade 3 to grade 2 or from grade 2 to grade 1.

4. A method of preventing complications of liver cirrhosis or all-cause mortality, or both, in a patient with liver decompensation comprising administering to the patient at least one rifaximin SSD IR tablet comprising about 40 mg of rifaximin.

5. The method of claim 4, wherein the complications of liver cirrhosis comprise one or more of hepatic encephalopathy (HE), esophageal variceal bleeding (EVB), spontaneous bacterial peritonitis (SBP), and hepatorenal syndrome (HRS).

6. The method of claim 4, wherein the patient has documented medically controlled non-refractory ascites.

7. The method of claim 4, wherein the patient has not previously experienced spontaneous bacterial peritonitis (SBP), esophageal variceal bleeding (EVB), or hepatorenal syndrome (HRS) prior to treatment.

8. The method of claim 4, wherein said method further comprises reducing the risk of hospitalizations associated with complications of liver cirrhosis or all-cause mortality, or both.

9. A method of delaying the onset of overt hepatic encephalopathy (OHE) in patients with liver decompensation comprising administering to the patient at least one rifaximin SSD IR tablet comprising about 40 mg of rifaximin.

10. The method of claim 9, wherein liver decompensation comprises cirrhosis of the liver and medically controlled ascites.

11. The method of claim 10, wherein the ascites are medically controlled for a period of at least 30 days.

12. The method of claim 10, wherein cirrhosis of the liver is determined by one or more of histopathological evidence of cirrhosis, transient elastography, the presence of esophageal varices, and thrombocytopenia characterized by a blood platelet level of less than 150,000 in a patient with chronic liver disease.

13. The method of claim 9, wherein the patient has a Child-Pugh B Classification score of from 7 to 9 without OHE prior to treatment.

14. The method of claim 9, wherein the patient has a Model End Stage Liver Disease-Sodium (MELD-Na) score of less than 15 prior to treatment.

15. The method of claim 9, wherein the patient has a Conn (West Haven Criteria) score of less than 2 prior to treatment.

16. The method of claim 9, wherein OHE comprises a Conn score of 2 or higher.

17. The method of claim 9, wherein the patient does not require paracentesis.

18. The method of claim 1, wherein the at least one rifaximin SSD IR tablet is administered to the patient for at least one day, or at least two days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days.

19. The method of claim 1, wherein the at least one rifaximin SSD IR tablet is administered to the patient once a day, twice a day, or three times a day.

20. The method of claim 1, wherein the method further comprises administering lactulose to the patient.
